(19) United States
(12) Patent Application Publication
(10) Pub. No.: US 2015/0209306 A1
(43) Pub. Date: Jul. 30, 2015

(54) MULTI-COMPONENT FORMULATION FOR IMPROVING NEUROLOGICAL FUNCTION

(71) Applicant: BUCK INSTITUTE FOR RESEARCH ON AGING, Novato, CA (US)

(72) Inventors: Dale E. Bredesen, Novato, CA (US); Stelios Tzannis, Petaluma, CA (US)

(21) Appl. No.: 14/418,402

(22) PCT Filed: Aug. 7, 2013
(86) PCT No.: PCT/US2013/053981

§ 371 Date: Jan. 29, 2015

Related U.S. Application Data
(60) Provisional application No. 61/680,653, filed on Aug. 7, 2012.

(51) Int. Cl. A61K 31/122; A61K 31/506; A61K 31/525; A61K 31/4406; A61K 31/164; A61K 31/4188; A61K 31/675; A61K 31/375; A61K 31/355; A61K 31/202; A61K 31/197; A61K 31/198; A61K 36/81; A61K 36/074; A61K 36/41; A61K 36/07; A61K 36/68; A61K 36/16; A61K 31/57; A61K 31/439

(52) U.S. Cl. CPC A61K 31/122; A61K 31/439; A61K 31/506; A61K 31/525; A61K 31/4406; A61K 31/164; A61K 31/4188; A61K 31/675; A61K 31/375; A61K 31/355; A61K 31/202; A61K 31/197; A61K 31/198; A61K 36/81; A61K 36/074; A61K 36/41; A61K 36/07; A61K 36/68; A61K 36/16; A61K 31/57

(57) ABSTRACT

In certain embodiments multi-component formulations are provided where the formulations comprise a first component comprising one or more vitamins selected from the group consisting of one or more B vitamins, vitamin C, vitamin D, vitamin E, co-enzyme Q10, vitamin K, and folate; a second component comprising one or more elements selected from the group consisting of selenium, lithium, magnesium, and molybdenum; a third component comprising one or more omega-3 fatty acids; and a fourth component comprising one or more amino acids selected from the group consisting of trimethylglycine, N-acetyl cysteine, S-adenosyl methionine, L-tryptophan, and glutathione.

### CROSS-REFERENCE TO RELATED APPLICATION

This application claims benefit under 35 U.S.C.§119(e) of U.S. provisional patent application No. 61/680,653 filed on Aug. 7, 2012, the contents of which is incorporated herein by reference in its entirety.

### STATEMENT OF GOVERNMENTAL SUPPORT

This work was supported in part by Grant No:AG034427 from the National Institute on Aging, National Institutes of Health. The Government has certain rights in this invention.

### BACKGROUND

The brain is a complex organ balancing numerous chemical pathways in order to preserve neuronal and synaptic function and overall brain health. Considerable research has been performed worldwide on the effects of aging and, in particular, neurological and neuropsychiatric diseases, on brain health and function. While much research has been focused on individual mechanisms in brain health using single agent pharmaceuticals or supplements, only a negligible fraction of the research efforts have addressed more than a single target at one time.

Several pharmaceutical candidates for the treatment of Alzheimer's disease (AD) have been developed by various researchers. However, to date, pharmaceuticals provide at most, only a short term benefit in neurological function.

### SUMMARY

Critical to brain health and wellness at any age would be healthy homeostatic levels of key moieties in the brain. Certain components are required to balance the numerous biochemical processes that take place in the brain at the cellular level. As such, the presence of these components can restore equilibria in brain and enhance neuronal function and boost all dependent processes, such as memory, cognition, etc. In addition, since these same biochemical pathways are shared in a number of diseases, such as Parkinson's disease, or deficiencies, such as memory reduction, one could in principle impact a number of medical needs.

Multi-component formulations are provided herein that find use, inter alia, in improving cognitive function in healthy individuals, in improving cognitive function or delaying or preventing a decline in cognitive function in subjects having or at risk for a neuropathology. In certain embodiments the multi-component formulation(s) restore healthy homeostatic levels of key moieties which is useful in preventing or reducing abnormalities associated with neurodegeneration. In certain embodiments the multi-component formulation(s) alone, or in combination with various active agents (e.g., as described herein) help prevent pre-symptomatic individuals from developing dementia or other neurodegenerative conditions. In various embodiments, the formulation(s) comprise a non-pharmaceutical supplement system that addresses key deficiencies in areas such as low endogenous growth factor levels, low anti-oxidant levels, high inflammation, low key vitamin levels, and low synaptic health constituents. Components are identified herein that are believed to achieve the highest possible impact on brain function/homeostasis via targeting multiple network components important in mediating neurodegeneration.

By virtue of their design, the multi-component formulations described herein are ideally positioned to improve neurological function in a subject. In particular, the multi-component formulations address the cognitive function decline in the elderly and in particular, those with early or established neuropsychiatric disease, such as those with mild cognitive impairment (referred to as MCI). Additionally, these formulations can address the need to improve memory in healthy individuals that would benefit from a boost of their memory and mental skill: professionals such as business executives, scientists, people generally on demanding assignments and even students, or simply those that want to maintain a high level of mental acuity.

In certain embodiments, a multi-component formulation is provided where the formulation comprises a first component comprising one or more vitamins selected from the group consisting of B vitamins, vitamin C, vitamin D, vitamin E (e.g., mixed tocopherols and tocotrienols), co-enzyme Q10, vitamin K, and folate; a second component comprising one or more elements selected from the group consisting of selenium, lithium, magnesium, and molybdenum; a third component comprising one or more omega-3 fatty acids; and a fourth component comprising one or more amino acids selected from the group consisting of trimethylglycine, N-acetyl cysteine, S-adenosyl methionine, L-tryptophan, and glutathione. In certain embodiments, the formulation further comprises a fifth component comprising one or more herbs selected from the group consisting of lion's main (Hericium), Bacopa monnieri, Ginkgo biloba, honokiol, magnolia extract, rosemary extract, ashwagandha, blueberry extract, bilberry extract, ginger, he shou wu, rhodiola, reishi, saffron, and daffodil. In certain embodiments any of these formulations further comprise a sixth component comprising one or more active agents selected from the group consisting of pregnenolone, galangin, vinpocetine, astaxanthin, and huperzine A. In certain embodiments any of these formulations further comprise a seventh component comprising a natural phenol. In certain embodiments any of these formulations further comprises an eighth component comprising a lipid or phospholipid. In certain embodiments any of these formulations further comprise a ninth component comprising a carbohydrate. In certain embodiments, the B vitamins comprise one or more vitamins selected from the group consisting of vitamin B1, vitamin B2, vitamin B3 (nicotinamide form), vitamin B5, vitamin B6, vitamin B7, vitamin B12, vitamin Bt (Carnitine), vitamin Benfotiamine, and vitamin Bx (PABA). In certain embodiments the vitamins comprise one or more vitamins selected from the group consisting of thiamine, nicotinamide, pantothenic acid, pyridoxal 5-phosphate, B12 (preferably hydroxocobalamin or methylcobalamin), vitamin C, vitamin E (mixed tocopherols and tocotrienols), vitamin K, and folate. In certain embodiments the vitamins comprise thiamine, nicotinamide, pantothenic acid, pyridoxal 5-phosphate, B12 (preferably hydroxocobalamin or methylcobalamin), vitamin C, vitamin E (mixed tocopherols and tocotrienols), vitamin K, and folate. In certain embodiments the vitamins comprise thiamine, nicotinamide, pantothenic acid, pyridoxine or pyridoxal 5-phosphate, B12 (preferably hydroxocobalamin or methylcobalamin), vitamin C, vitamin E (mixed tocopherols and tocotrienols), vitamin K, and folate. In certain embodiments of any of these formulations one or more elements are present and comprise comprises lithium. In certain embodiments of any of these formulations the omega-3 fatty acid comprises one or more a fatty acids selected from the group consisting of docosahexaenoic acid, and eicosapentaenoic acid. In certain embodiments of any of these formulations the omega-3 fatty acid comprises docosahexaenoic acid. In certain embodiments of any of these formulations the one or more amino acids comprise one or more amino acids selected from the group consisting of trimethyl glycine, N-acetyl cysteine, and S-adenosyl methionine. In certain embodiments of any of these formulations the one or more amino acids comprise trimethyl glycine, N-acetyl cysteine, and S-adenosyl methionine. In certain embodiments of any of these formulations the one or more herbs comprise one or more herbs selected from the group consisting of Withania somnifera (ashwagandha), Reishi, Rhodiola, Lion's Mane (Hericium Erinaceous), Bacopa monnieri, Ginkgo biloba, Honokiol, and ginger. In certain embodiments of any of these formulations the one or more herbs comprise Lion's Mane (Hericium erinaceus), Bacopa monnieri, Ginkgo biloba, Withania somnifera (ashwagandha), Reishi, Rhodiola, Honokiol, and Ginger. In certain embodiments of any of these formulations the one or more active agents comprise one or more active agents selected from the group consisting of pregnenolone, and galangin. In certain embodiments of any of these formulations the active agents comprise pregnenolone, and galangin. In certain embodiments of any of these formulations the natural phenols, when present, comprise a cucuminoid. In certain embodiments of any of these formulations the natural phenols comprise cucumin and/or turmeric. In certain embodiments of any of these formulations the lipid or phospholipid comprise one or more lipids or phospholipids selected from the group consisting of CDP-choline, Phosphatidyl choline, Choline, Phosphatidyl Serine, and Lipoic Acid. In certain embodiments of any of these formulations the lipid or phospholipid comprises choline. In certain embodiments the carbohydrate comprises inositol. In certain embodiments the formulation comprises at least four agents selected from the group consisting of vitamin B1, vitamin B5, nicotinamide, vitamin B6, vitamin B12, carnitine, vitamin C, vitamin D, vitamin E, vitamin K, folate, selenium, lithium, Docosahexaenoic Acid, eisopantaenoic acid, choline, Trimethylglycine, L-Tryptophan, N-Acetyl-Cysteine, S-Adenosyl Methionine (SAMe), Melatonin, Pregnenolone, Galangin, Lion's Mane (Hericium Erinaceous), Bacopa monnieri, Ginkgo biloba, Withania somnifera (ashwagandha), Reishi, Rhodiola, Honokiol, and ginger, wherein said at least four different agents comprise at least four different components. In certain embodiments formulation comprises at least five different agents selected from said group and said at least five different agents comprise at least five different components. In certain embodiments the formulation comprises at least six different agents selected from said group and said at least six different agents comprise at least six different components. In certain embodiments the formulation comprises at least seven different agents selected from said group and said at least seven different agents comprise at least seven different components. In certain embodiments the formulation comprises at least eight different agents selected from said group and said at least eight different agents comprise at least eight different components. In certain embodiments the formulation comprises: said first component wherein said first component comprises vitamin B1, and/or vitamin B5, and/or nicotinamide and/or vitamin B6, and/or vitamin B12, and/or carnitine, and/or vitamin C, and/or vitamin E, and/or vitamin K, and/or folate; said second component wherein said second component comprises selenium and/or lithium; said third component wherein said third component comprises an omega-3 fatty acid; said fourth component wherein said fourth component comprises trimethylglycine, and/or N-acetyl cysteine, and/or S-adenosyl methionine; said fifth component wherein said fifth component comprises Lion's Mane, and/or Bacopa monnieri, and/or Ginkgo biloba, and/or Withania somnifera (ashwagandha), and/or Reishi, and/or Rhodiola, and/or Honokiol; and said sixth component wherein said sixth component comprises pregnenolone, and/or galangin. In certain embodiments the first component comprises vitamin B1, vitamin B5, nicotinamide, vitamin B6, vitamin B12, carnitine, vitamin C, vitamin E, vitamin K, and folate; the second component comprises selenium and/or lithium; the third component comprises docosahexaenoic acid, and/or eisopentanoic Acid; the fourth component comprises trimethylglycine, N-acetyl cysteine, and S-adenosyl methionine; the fifth component comprises Lion's Mane, Bacopa monnieri, Ginkgo biloba, Withania somnifera (ashwagandha), Reishi, Rhodiola, and Honokiol; and the sixth component comprises melatonin, pregnenolone, and galangin. In certain embodiments this formulation further comprises said seventh component, wherein said seventh component comprises a cucuminoid. In certain embodiments the formulation further comprises said eighth component, wherein said eighth component comprises a lipid or phospholipid. In certain embodiments the lipid or phospholipid comprises choline. In certain embodiments the formulation further comprises said ninth component, wherein said ninth component comprises inosotol. In certain embodiments vitamin B1, when present, comprises at least about 2.5 mg; nicotinamide, when present, comprises at least 50 mg; vitamin B5, when present, comprises at least 50 mg; vitamin B6, when present, comprises at least 5 mg; vitamin B12, when present, comprises at least about 0.1 mg; carnitine, when present, comprises at least about 100 mg; vitamin C, when present, comprises at least about 100 mg; vitamin D, when present, comprises at least about 1000 IU; vitamin E, when present, comprises at least about 50 mg; vitamin K, when present, comprises at least about 10 mg; folate, when present, comprises at least about 0.2 mg; selenium, when present, comprises at least about 25 μg; lithium, when present, comprises at least about 1 mg; inosotol, when present, comprises at least about 500 mg; docosahexaenoic acid, when present, comprises at least about 0.25 g; eicosapentanoic acid, when present, comprises at least about 0.25 g; choline, when present, comprises at least about 0.5 g; trimethylglycine, when present, comprises at least about 120 mg; N-acetyl-cysteine, when present, comprises at least about 200 mg; S-adenosyl methionine, when present, comprises at least about 100 mg; a curcuminoid, when present, comprises at least about 250 mg; pregnenolone, when present, comprises at least about 2 mg; galangin, when present, comprises at least about 200 mg; Lion's Mane, when present, comprises at least about 250 mg; Bacopa monnieri, when present, comprises at least about 50 mg; Ginkgo biloba, when present, comprises at least about 20 mg; Honokiol, when present, comprises at least about 200 mg; and Ginger, when present, comprises at least about 100 mg. In certain embodiments vitamin B1, when present, ranges from about 100 to about 750 mg; vitamin B5, when present, ranges from about 25 to about 150 mg; vitamin B6, when present, ranges from about 5 to about 50 mg; vitamin B12, when present, ranges from about 0.1 mg to about 3 mg; acetyl-L-carnitine (ALCAR), when present, ranges from about 250 mg to about 2000 mg; vitamin C, when present, ranges from about 100 mg to about 1000 mg vitamin D, when present, ranges from about 1000 IU to about 4000 IU; vitamin E, when present, ranges from about 50 mg to about 1500 mg; vitamin K, when present, ranges from about 10 mg to about 200 mg; folate, when present, ranges from about 0.2 mg to about 1.5 mg; selenium, when present, ranges from about 25 μg to about 500 μg; lithium, when present, ranges from about 1 mg to about 20 mg; inosotol, when present, ranges from about 0.25 mg to about 1.5 mg; docosahexaenoic acid, when present, ranges from about 0.25 g to about 1.5 g; eicosapentaenoic, when present, ranges from about 0.25 g to about 1.5 g; choline, when present, ranges from about 0.5 g to about 3 g; trimethylglycine, when present, ranges from about 120 mg to about 1000 mg; N-acetyl-cysteine, when present, ranges from about 200 mg to about 1000 mg; S-adenosyl methionine, when present, ranges from about 100 mg to about 600 mg; a curcuminoid, when present, ranges from about 500 mg to about 4000 mg; pregnenolone, when present, ranges from about 2 mg to about 5 mg; galangin, when present, ranges from about 200 mg to about 8000 mg; Lion's Mane, when present, ranges from about 250 mg to about 2000 mg; Bacopa monnieri, when present, ranges from about 50 mg to about 600 mg; Ginkgo biloba, when present, ranges from about 20 mg to about 200 mg; Honokiol, when present, ranges from about 1 mg to about 1000 mg active ingredient; and Ginger, when present, ranges from about 100 mg to about 1000 mg. In certain embodiments vitamin B1 is present and ranges from about 100 to about 750 mg; vitamin B5 is present and ranges from about 25 to about 150 mg; vitamin B6 is present and ranges from about 5 to about 50 mg; vitamin B12 is present and ranges from about 0.1 mg to about 3 mg; acetyl-L-carnitine (ALCAR) is present and ranges from about 250 mg to about 2000 mg; vitamin C is present and ranges from about 100 mg to about 1000 mg; vitamin D is present and ranges from about 1000 IU to about 4000 IU; vitamin E is present and ranges from about 50 mg to about 1500 mg; vitamin K is present and ranges from about 10 mg to about 200 mg; folate is present and ranges from about 0.2 mg to about 1.5 mg; selenium is present and ranges from about 25 μg to about 500 μg; lithium is present and ranges from about 1 mg to about 20 mg; inosotol is present and ranges from about 0.25 mg to about 1.5 mg; docosahexaenoic acid is present and ranges from about 0.25 g to about 1.5 g; eicosapentaenoic acid is present and ranges from about 0.25 g to about 1.5 g; choline is present and ranges from about 0.5 g to about 3 g; trimethylglycine is present and ranges from about 120 mg to about 1000 mg; N-acetyl-cysteine is present and ranges from about 200 mg to about 1000 mg; S-adenosyl methionine is present and ranges from about 100 mg to about 600 mg; a curcuminoid is present and ranges from about 500 mg to about 4000 mg; pregnenolone is present and ranges from about 2 mg to about 5 mg; galangin is present and ranges from about 200 mg to about 1000 mg; Lion's Mane is present and ranges from about 250 mg to about 2000 mg; Bacopa monnieri is present and ranges from about 50 mg to about 600 mg; Ginkgo biloba is present and ranges from about 20 mg to about 200 mg; Honokiol is present and ranges from about 1 mg to about 1000 mg; and Ginger is present and ranges from about 100 mg to about 1000 mg. In certain embodiments the components are contained in single packaging system. In certain embodiments two or more of said components are encapsulated in separate capsules, vials, or tablets. In certain embodiments the fluid components are encapsulated separately from solid components. In certain embodiments all of the components are provided in a single combined formulation.

In certain embodiments methods of slowing the rate of decrease in neurological function, or delaying the onset of a decrease in neurological function, in a mammal are provided. The methods typically comprise administering to a mammal in need thereof a multi-component formulation as described herein in an amount sufficient to slow the rate of decrease in neurological function or to delay the onset of a decrease in neurological function in said mammal. In certain embodiments the mammal is a mammal that has a neurological disorder. In certain embodiments the mammal is a mammal that has been identified as at risk for a neurological disorder. In certain embodiments the mammal is a normal healthy mammal and said decrease in neurological function is an age related decrease in neurological function. In certain embodiments the mammal is a normal healthy mammal and said decrease in neurological function is a stress-induced decrease in neurological function.

In various embodiments methods are also provided for improving neurological function or in a mammal. These methods typically comprise administering or causing to be administered to the mammal (e.g., to a mammal in need thereof) a multi-component formulation as described and/or claimed herein in an amount sufficient to improve neurological function. In certain embodiments the mammal is a mammal that has a neurological disorder. In certain embodiments the mammal is a mammal that has been identified as at risk for a neurological disorder. In certain embodiments the mammal is a mammal with no neurological disorder.

Methods are also provided for normalizing neurological function to optimize treatment for a neurological disorder in a mammal. These methods typically comprise administering or causing to be administered to the mammal (e.g., to a mammal in need thereof) a multi-component formulation as described and/or claimed herein in an amount sufficient to improve cognitive function as measured by a standard neuropsychological cognitive test in a subject with abnormal cognition or in a subject with normal cognition; and/or to prevent or delay progression of symptoms of neurodegeneration.

In various embodiments methods are also provided for preventing or delaying the onset of a pre-Alzheimer's condition and/or cognitive dysfunction, and/or for ameliorating one or more symptoms of a pre-Alzheimer's condition and/or cognitive dysfunction, and/or preventing or for delaying the progression of a pre-Alzheimer's condition or cognitive dysfunction to Alzheimer's disease in a mammal. These methods typically comprise administering or causing to be administered to the mammal (e.g., to a mammal in need thereof) a multi-component formulation as described and/or claimed herein in an amount sufficient to to slow the rate of decrease in neurological function or to prevent or delay the onset of a pre-Alzheimer's condition and/or cognitive dysfunction, and/or to ameliorate one or more symptoms of a pre-Alzheimer's condition and/or cognitive dysfunction, and/or to prevent or delay the progression of a pre-Alzheimer's condition or cognitive dysfunction to Alzheimer's disease in said mammal. In various embodiments of any of these methods the neurological function can comprise one or more functions selected from the group consisting of memory, cognition, concentration, gross motor control, and fine motor control. In various embodiments of any of these methods an improvement in neurological function can be characterized by, or associated with, a reduction in the mammal's CSF of levels of one or more components selected from the group consisting of total-Tau (tTau), phospho-Tau (pTau), APPneo, soluble Aβ40, pTau/Aβ42 ratio and tTau/Aβ42 ratio, and/or an increase in the mammal's CSF of levels of one or more components selected from the group consisting of Aβ40/Ab42 ratio, Aβ38/Ab42 ratio, sAPPα, sAPPα/sAPPβ ratio, sAPPα/Aβ40 ratio, or sAPPα/Aβ42 ratio. In various embodiments of any of these methods, the rate of a decrease in neurological function can be characterized by, or associated with, a stabilization or a reduction in the mammal's CSF of levels of one or more components selected from the group consisting of total-Tau (tTau), phospho-Tau (pTau), APPneo, soluble Aβ40, pTau/Aβ42 ratio and tTau/Aβ42 ratio, and/or a stabilization or an increase in the mammal's CSF of levels of one or more components selected from the group consisting of Aβ40/Ab42 ratio, Aβ38/Ab42 ratio, sAPPα, sAPPα/sAPPβ ratio, sAPPα/Aβ40 ratio, or sAPPα/Aβ42 ratio. In certain embodiments, of any of these methods, all components of the multi-component formulation are administered to the mammal at least once a week, or at least twice a week, or at least every other day, or at least once a day, or at least 2, or at least 3 or at least 4 times daily. In certain embodiments, of any of these methods, all components of the multi-component formulation are administered to the mammal at least once a day. In certain embodiments of any of these methods, the mammal is diagnosed with a neurological disorder selected from the group consisting of pre-Alzheimer's disease, mild cognitive impairment, early stage Alzheimer's disease, late stage Alzheimer's disease, age-related dementia, Parkinson's disease, Huntington's disease, Multiple Sclerosis, Amyotrophic Lateral Sclerosis (ALS or Lou Gehrig's Disease), Prion Diseases, Creutzfeldt-Jakob disease, Lewy Body disease, Friedreich's Ataxia, Stroke, Genetic Brain Disorders, Schizophrenia, ADHD, Autism, Aspergers syndrome, and Downs syndrome. In certain embodiments of any of these methods, the mammal is determined to be at risk for a neurological disorder selected from the group consisting of pre-Alzheimer's disease, mild cognitive impairment, early stage Alzheimer's disease, late stage Alzheimer's disease, age-related dementia, Parkinson's disease, Huntington's disease, Multiple Sclerosis, Amyotrophic Lateral Sclerosis (ALS or Lou Gehrig's Disease), Prion Diseases, Creutzfeldt-Jakob disease, Lewy Body disease, Friedreich's Ataxia, Stroke, Genetic Brain Disorders, Schizophrenia, ADHD, Autism, Aspergers syndrome, and Downs syndrome. In certain embodiments of these methods, the neurological disorder comprises a pre-Alzheimer's neurological and/or cognitive dysfunction. In certain embodiments of these methods, the neurological disorder comprises MCI. In certain embodiments of these methods, the neurological disorder comprises Alzheimer's disease. In various embodiments of any of these methods, the mammal is a human. In certain embodiments of these methods, the mammal is a human diagnosed as having or as at risk for the neurological disorder. In certain embodiments of these methods, the mammal is a human diagnosed as having or as at risk for MCI. In certain embodiments of these methods, the mammal is a human diagnosed as having or as at risk for Alzheimer's disease.

In certain embodiments methods of enhancing the efficacy of an agent the treatment and/or prophylaxis of a neurological disorder in a mammal are provided. In various embodiments the methods typically comprise administering, or causing to be administered, in conjunction with the agent, a multi-component formulation as described and/or claimed herein. In certain embodiments, of any of these methods, all components of the multi-component formulation are administered to the mammal at least once a week, or at least twice a week, or at least every other day, or at least once a day, or at least 2, or at least 3 or at least 4 times daily. In certain embodiments, of any of these methods, all components of the multi-component formulation are administered to the mammal at least once a day. In certain embodiments, the neurological disorder comprises a disorder selected from the group consisting pre-Alzheimer's disease, mild cognitive impairment, early stage Alzheimer's disease, late stage Alzheimer's disease, age-related dementia, Parkinson's disease, Huntington's disease, Multiple Sclerosis, Amyotrophic Lateral Sclerosis (ALS or Lou Gehrig's Disease), Prion Diseases, Creutzfeldt-Jakob disease, Lewy Body disease, Friedreich's Ataxia, Stroke, Genetic Brain Disorders, Schizophrenia, ADHD, Autism, Aspergers syndrome, and Downs syndrome. In certain embodiments the neurological disorder comprises MCI or another pre-Alzheimer's condition. In certain embodiments the neurological disorder comprises Alzheimer's disease. In certain embodiments the mammal is a human. In certain embodiments the mammal is a human diagnosed as having or as at risk for the neurological disorder (e.g., a human diagnosed as having or as at risk for MCI, a human diagnosed as having or as at risk for Alzheimer's disease, a human diagnosed as having or at risk for age-related dementia, etc.). In certain embodiments the agent comprises a therapeutic or prophylactic agent selected from the group consisting of a tropisetron analog, disulfiram, a disulfiram analog, honokiol, a honokiol analog, nimetazepam, a nimetazepam analog, tropinol-esters, ADDN-1351, TrkA kinase inhibitors, donepezil, rivastigmine, galantamine, tacrine, memantine, solanezumab, bapineuzmab, alzemed, flurizan, ELND005, valproate, semagacestat, rosiglitazone, phenserine, cernezumab, dimebon, egcg, gammagard, PBT2, PF04360365, NIC5-15, bryostatin-1, AL-108, nicotinamide, EHT-0202, BMS708163, NP12, lithium, ACC001, AN1792, ABT089, NGF, CAD106, AZD3480, SB742457, AD02, huperzine-A, EVP6124, PRX03140, PUFA, HF02, MEM3454, TTP448, PF-04447943, GSK933776, MABT5102A, talsaclidine, UB311, begacestat, R1450, PF3084014, V950, E2609, MK0752, CTS21166, AZD-3839, LY2886721, CHF5074, an anti-inflammatory, dapsone, an anti-TNF antibody, and a statin. In certain embodiments the agent is tropisetron or an analog thereof. In certain embodiments the agent is tropisetron. In certain embodiments the agent is a tropinol ester.

In various embodiments methods for the treatment or prophylaxis of a neurological/neurodegenerative disorder in a mammal are provided. In certain embodiments the methods typically comprise administering, or causing to be administered, to a mammal in need thereof one or more agents for the treatment or prophylaxis of a neurological disorder; and a multi-component formulation as described and/or claimed herein. In certain embodiments of these methods, all components of the multi-component formulation are administered to the mammal at least once a week, or at least twice a week, or at least every other day, or at least once a day, or at least 2, or at least 3 or at least 4 times daily. In certain embodiments of these methods, all components of the multi-component formulation are administered to the mammal at least once a day. In certain embodiments the neurological (and/or neurodegenerative) disorder comprises a disorder selected from the group consisting of pre-Alzheimer's disease, mild cognitive impairment, early stage Alzheimer's disease, late stage Alzheimer's disease, age-related dementia, Parkinson's disease, Huntington's disease, Multiple Sclerosis, Amyotrophic Lateral Sclerosis (ALS or Lou Gehrig's Disease), Prion Diseases, Creutzfeldt-Jakob disease, Lewy Body disease, Friedreich's Ataxia, Stroke, Genetic Brain Disorders, Schizophrenia, ADHD, Autism, Aspergers syndrome, and Downs syndrome. In certain embodiments the neurological disorder comprises pre-Alzheimer's disease. In certain embodiments the neurological disorder comprises MCI. In certain embodiments the neurological disorder comprises Alzheimer's disease. In certain embodiments the mammal is a human. In certain embodiments the mammal is a human having or as at risk for MCI. In certain embodiments the administration delays or prevents the progression of MCI to Alzheimer's disease. In certain embodiments the mammal is at risk of developing Alzheimer's disease. In certain embodiments the mammal has a familial risk for having Alzheimer's disease. In certain embodiments the mammal has a familial Alzheimer's disease (FAD) mutation. In certain embodiments the mammal has the APOE ε4 allele. In certain embodiments mammal is free of and does not have genetic risk factors of Parkinson's disease or schizophrenia. In certain embodiments the mammal is not diagnosed as having or at risk for Parkinson's disease or schizophrenia. In certain embodiments the mammal does not have a neurological disease or disorder other than Alzheimer's disease. In certain embodiments the mammal is not diagnosed as having or at risk for a neurological disease or disorder other than Alzheimer's disease. In certain embodiments the mammal does not have a neurological disease or disorder other than MCI. In certain embodiments the mammal is not diagnosed as having or at risk for a neurological disease or disorder other than MCI. In various embodiments of any of these methods the method(s) result in a reduction in the CSF of levels of one or more components selected from the group consisting of total-Tau (tTau), phospho-Tau (pTau), APPneo, soluble Aβ40, pTau/Aβ42 ratio and tTau/Aβ42 ratio, and/or an increase in the CSF of levels of one or more components selected from the group consisting of Aβ40/Aβ42 ratio, Aβ38/Aβ42 ratio, sAPPα, sAPPα/sAPPβ ratio, sAPPα/Aβ40 ratio, and sAPPα/Aβ42 ratio. In certain embodiments the method(s) produce a reduction of the plaque load in the brain of the mammal. In certain embodiments the method(s) produce a reduction in the rate of plaque formation in the brain of the mammal. In certain embodiments the method provides an improvement in the cognitive abilities of the mammal. In certain embodiments the method produces an improvement in, a stabilization of, or a reduction in the rate of decline of the clinical dementia rating (CDR) of the mammal. In certain embodiments the mammal is a human and the method produces a perceived improvement in quality of life by the human. In certain embodiments the administering is over a period of at least three weeks, or over a period of at least six weeks, or over a period of at least two months, or over a period of at least four months, or over a period of at least six months, or over a period of at least one year, or over a period of at least two years, or over a period of at least three years, or over a period of at least five years, or over a period of at least ten years. In certain embodiments the mammal is a human diagnosed as having or as at risk for the neurological disorder (e.g., a human diagnosed as having or as at risk for MCI, a human diagnosed as having or as at risk for Alzheimer's disease, a human diagnosed as having or at risk for age-related dementia, etc.). In certain embodiments the agent comprises a therapeutic or prophylactic agent selected from the group consisting of a tropisetron analog, disulfiram, a disulfiram analog, honokiol, a honokiol analog, nimetazepam, a nimetazepam analog, tropinol-esters, ADDN-1351, TrkA kinase inhibitors, donepezil, rivastigmine, galantamine, tacrine, memantine, solanezumab, bapineuzmab, alzemed, flurizan, ELND005, valproate, semagacestat, rosiglitazone, phenserine, cernezumab, dimebon, egcg, gammagard, PBT2, PF04360365, NIC5-15, bryostatin-1, AL-108, nicotinamide, EHT-0202, BMS708163, NP12, lithium, ACC001, AN1792, ABT089, NGF, CAD106, AZD3480, SB742457, AD02, huperzine-A, EVP6124, PRX03140, PUFA, HF02, MEM3454, TTP448, PF-04447943, GSK933776, MABT5102A, talsaclidine, UB311, begacestat, R1450, PF3084014, V950, E2609, MK0752, CTS21166, AZD-3839, LY2886721, CHF5074, an anti-inflammatory, dapsone, an anti-TNF antibody, and a statin. In certain embodiments the agent is tropisetron or an analog thereof. In certain embodiments the agent is tropisetron. In certain embodiments the agent is a tropinol ester. In various embodiments of these methods, an acetylcholinesterase inhibitor (e.g., tacrine-ipidacrine, galantamine, donepezil, icopezil, zanapezil, rivastigmine, Namenda, huperzine A, phenserine, physostigmine, neostigmine, pyridostigmine, ambenonium, demarcarium, edrophonium, ladostigil and ungeremine and metrifonate, etc.) is not administered in conjunction with said multi-component formulation.

In various embodiments kits are also provided for the treatment or prophylaxis of a neurological disorder and/or for the maintenance or improvement of cognitive health. In certain embodiments the kits can comprise a packaging system containing a multi-component formulation described and/or claimed herein. In certain embodiments the kit additionally comprises one or more agents for the treatment or prophylaxis of a neurological disorder. In certain embodiments the components of the multi-component formulation are contained in a first packaging system and the one or more agents are contained in a second packaging system. In certain embodiments two or more of the components of the multi-component formulation components are encapsulated in separate capsules, vials, or tablets. In certain embodiments fluid components of the multi-component formulation are encapsulated separately from solid components. In certain embodiments all of the components of the multi-component formulation are provided in a single combined formulation. In certain embodiments the agent comprises a therapeutic or prophylactic agent selected from the group consisting of a tropisetron analog, disulfiram, a disulfiram analog, honokiol, a honokiol analog, nimetazepam, a nimetazepam analog, tropinol-esters, ADDN-1351, TrkA kinase inhibitors, donepezil, rivastigmine, galantamine, tacrine, memantine, solanezumab, bapineuzmab, alzemed, flurizan, ELND005, valproate, semagacestat, rosiglitazone, phenserine, cernezumab, dimebon, egcg, gammagard, PBT2, PF04360365, NIC5-15, bryostatin-1, AL-108, nicotinamide, EHT-0202, BMS708163, NP12, lithium, ACC001, AN1792, ABT089, NGF, CAD106, AZD3480, SB742457, AD02, huperzine-A, EVP6124, PRX03140, PUFA, HF02, MEM3454, TTP448, PF-04447943, GSK933776, MABT5102A, talsaclidine, UB311, begacestat, R1450, PF3084014, V950, E2609, MK0752, CTS21166, AZD-3839, LY2886721, CHF5074, an anti-inflammatory, dapsone, an anti-TNF antibody, and a statin. In certain embodiments the agent is tropisetron or an analog thereof. In certain embodiments the agent is tropisetron. In certain embodiments the agent is a tropinol ester.

### DEFINITIONS

As used herein, the term “neurological disorder” refers to disorders of the central and peripheral nervous system, e.g., the brain, spinal cord, cranial nerves, peripheral nerves, nerve roots, autonomic nervous system, neuromuscular junction, and muscles. Various neurological disorders affect the structure, biochemical, and/or electrical systems in the brain, spinal cord or other nerves and can result in a range of symptoms. Examples of symptoms include paralysis, muscle weakness, poor coordination, loss of sensation, seizures, confusion, memory loss, pain and altered levels of consciousness. In general, neurological disorders may be assessed by neurological examination, and studied and treated within the specialties of neurology and clinical neuropsychology. Neurological disorders include neurodegenerative disorders.

As used herein, the phrase “neurodegenerative disorder” refers to any disorder, disease or condition of the nervous system (typically CNS) that is characterized by gradual and progressive loss of neural tissue, neurotransmitter, or neural functions. Examples of neurodegenerative disorders include, but are not limited to, Parkinson's disease, Alzheimer's disease, frontotemporal dementia, vascular dementia, age-related dementia, glaucomatous neuropathy, autoimmune encephalomyelitis, diabetic neuropathy, cerebrovascular accident (stroke), idiopathic dementia, Huntington's disease, mild cognitive impairment (MCI), multiple sclerosis, amyotrophic lateral sclerosis (ALS or Lou Gehrig's Disease), prion diseases, Creutzfeldt-Jakob disease, Lewy body disease, Friedreich's ataxia, stroke, genetic brain disorders, progressive supranuclear palsy (PSP), and the like.

Vitamins as used herein, unless indicated otherwise, include both the natural form of the vitamin and/or synthetic forms. Thus for example, “vitamin C” refers to ascorbic acid, which is an essential nutrient found in fruit and vegetables. Vitamin C includes the synthetic or natural form of vitamin C, such as the vitamin C extracted from corn syrup or sago palm. Vitamin C also includes the vitamin extracted from other natural sources such as for example rose hips, acerola cherries, peppers, or citrus fruits. Vitamin C also refers to mineral ascorbates (such as sodium, potassium, calcium, zinc, molybdenum, chromium and manganese ascorbates), ascorbyl palmitate and D-isoascorbic acid. Similarly, “vitamin E” refers to any one or combination of the eight forms of vitamin E comprising the four tocopherols (α, β, γ, δ) and the four tocotrienols (α, β, γ, δ) including the succinate, nicotinate and acetate salts derivatives thereof. In addition, each of these compounds has a “d” form, which is the natural form, and a “dl” form, which is the synthetic form.

An “herb” refers to a fresh or dried part of a plant or a whole plant or an extract thereof, that comprises biological activity (e.g., for the normalization of neurological function as described herein). Thus, for example, “Ginkgo biloba” refers to the active ingredients extracted from the Ginkgo biloba tree including ginkgoflavoneglycos, bilobalide, and terpenelactones including ginkgolides A, B and C or plant portions thereof. One example of a standardized extract is EGb761 (Natures Way, U.S.A.) comprising approximately 24% flavone glycosides (primarily quercetin, kaempferol and isorhamnetin) and 6% terpene lactones (2.8-3.4% ginkgolides A, B and C, and 2.6-3.2% bilobalide). Ginkgolide B and bilobalide account for about 0.8% and 3% of the total extract, respectively. Other constituents include proanthocyanadins, glucose, rhamnose, organic acids, D-glucaric and ginkgolic acids. Other examples of standardized Ginkgo biloba extracts include, but are not limited to the three formulations which are available from Linnea (Switzerland) (EPG 246: 24% ginkgo flavonglycosides, 6% terpene lactones; G 328: 32% ginkgo flavonglycosides, 8% terpene lactones; G 320: 32% ginkgo flavonglycosides, without terpene lactones), and the like.

As used herein, the phrase “a subject in need ‘thereof” refers to a subject, as described infra, that suffers or is at a risk of suffering (i.e., pre-disposed such as genetically pre-disposed) from the diseases or conditions listed herein.

The term “co-administering” or “concurrent administration” or “administering in conjunction with” when used, for example with respect to the multi-component formulation(s) described herein and a composition comprising one or more pharmaceuticals or other active agents (e.g., tropisetron or other tropinol esters, honokiol, disulfram, nimetazepam, ADDN-1351, TrkA kinase inhibitors, D2 receptor agonists, alpha1-adrenergic receptor antagonists, and/or analogues or derivatives thereof), refers to administration of the multi-component formulation and the composition such that both can simultaneously achieve a physiological effect. The multi-component formulation and the active agent composition, however, need not be administered together, either temporally or at the same site; moreover, the multi-component formulation and the composition need not be administered by the same method, e.g., the multi-component formulation may be administered orally and the composition may be administered intravenously or orally. In a particular embodiment, the multi-component formulation and the active agent composition are administered at different times and by different methods of administration. In certain embodiments, administration of one can precede administration of the other. Simultaneous physiological effect need not necessarily require presence the multi-component formulation and the active agent composition in the circulation at the same time. However, in certain embodiments, co-administering typically results in both the multi-component formulation and the composition being simultaneously present in the body (e.g., in the plasma) at a significant fraction (e.g., 20% or greater, preferably 30% or 40% or greater, more preferably 50% or 60% or greater, most preferably 70% or 80% or 90% or greater) of their maximum serum concentration for any given dose.

The terms “subject,” “individual,” and “patient” may be used interchangeably and refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child) under the care of a physician or other health worker in a hospital, psychiatric care facility, as an outpatient, or other clinical context. In certain embodiments, the subject may not be under the care or prescription of a physician or other health worker.

An “effective amount” refers to an amount effective, at dosages and for periods of time necessary, to achieve the desired therapeutic or prophylactic result. A “therapeutically effective amount” of a multi-component formulation, optionally in combination with one or more pharmaceuticals, may vary according to factors such as the disease state, age, sex, and weight of the individual, the pharmaceutical (and dose thereof) when used in combination with pharmaceutical, and the ability of the treatment to elicit a desired response in the individual. A therapeutically effective amount is also one in which any toxic or detrimental effects of a treatment are substantially absent or are outweighed by the therapeutically beneficial effects. The term “therapeutically effective amount” refers to an amount of an active agent or composition comprising the same that is effective to “treat” a disease or disorder in a mammal (e.g., a patient). In one embodiment, a therapeutically effective amount is an amount sufficient to improve at least one symptom associated with a neurological disorder, improve neurological function, improve cognition, or one or more markers of a neurological disease, or to enhance the efficacy of one or more pharmaceuticals administered for the treatment or prophylaxis of a neurodegenerative pathology. In certain embodiments, an effective amount is an amount sufficient alone, or in combination with a pharmaceutical agent to prevent advancement or the disease, delay progression, or to cause regression of a disease, or which is capable of reducing symptoms caused by the disease,

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount is less than the therapeutically effective amount.

The terms “treatment,” “treating,” or “treat” as used herein, refer to actions that produce a desirable effect on the symptoms or pathology of a disease or condition, particularly those that can be effected utilizing the multi-component formulation(s) described herein, and may include, but are not limited to, even minimal changes or improvements in one or more measurable markers of the disease or condition being treated. Treatments also refers to delaying the onset of, retarding or reversing the progress of, reducing the severity of, or alleviating or preventing either the disease or condition to which the term applies, or one or more symptoms of such disease or condition. “Treatment,” “treating,” or “treat” does not necessarily indicate complete eradication or cure of the disease or condition, or associated symptoms thereof. In one embodiment, treatment comprises improvement of at least one symptom of a disease being treated. The improvement may be partial or complete. The subject receiving this treatment is any subject in need thereof. Exemplary markers of clinical improvement will be apparent to persons skilled in the art.

The term “mitigating” refers to reduction or elimination of one or more symptoms of that pathology or disease, and/or a reduction in the rate or delay of onset or severity of one or more symptoms of that pathology or disease, and/or the prevention of that pathology or disease.

As used herein, the phrases “improve at least one symptom” or “improve one or more symptoms” or equivalents thereof, refer to the reduction, elimination, or prevention of one or more symptoms of pathology or disease. Illustrative symptoms of pathologies treated, ameliorated, or prevented by the compositions and/or formulations described herein include, but are not limited to, reduction, elimination, or prevention of one or more markers that are characteristic of the pathology or disease (e.g., of total-Tau (tTau), phospho-Tau (pTau), APPneo, soluble Aβ40, pTau/Ap42 ratio and tTau/Ap42 ratio, and/or an increase in the CSF of levels of one or more components selected from the group consisting of Aβ42/Aβ40 ratio, Aβ42/Aβ38 ratio, sAPPα, βAPPα/βAPPβ ratio, βAPPα/Aβ40 ratio, βAPPα/Aβ42 ratio, etc.) and/or reduction, stabilization or reversal of one or more diagnostic criteria (e.g., clinical dementia rating (CDR)). Illustrative measures for improved neurological function include, but are not limited to the use of the mini-mental state examination (MMSE) or Folstein test (a questionnaire test that is used to screen for cognitive impairment), the General Practitioner Assessment of Cognition (GPCOG), a brief screening test for cognitive impairment described by Brodaty et al. (2002) Geriatrics Society 50(3): 530-534, and the like.

### BRIEF DESCRIPTION OF THE DRAWINGS

FIG. 1 shows one exemplary SYNAPTIK™ formulation incorporating vitamins (vitamin B1, vitamin B3 (niacinamide), vitamin B5 (PA), vitamin B6 (P5P), methyl (MTH) folate, methyl B12, ALCAR (acetyl caranitine), vitamin E, vitamin C, vitamin D3), carbohydrates (inositol), amino acids (trimethylglycine, N-acetyl cysteine (NAC), and S-adenosyl methionine), omega-3 fatty acids (DHA and EPA), lipid/phospholipid (citicoline), a phenol (curcumin), and various herbs (herbs (e.g., Bacopa monnieri, lion's mane, Ginkgo biloba (phytosome complex), and ginger) that is achieved with a combination of commercially available supplements (e.g., PURITANS PRIDE Mega B-150, THORNE Neurochondria, THORNE B12 complex, SOURCE NATURALS (BIOVEA), PURITAN'S PRIDE omega-3 fish oil+vitamin D, THORNE MEMORACTIVE, LIFE EXTENSION super curcumin+bioperine, HEALTHY ORIGINS cognizin citicoline (evidencia), PURITAN'S PRIDE C-500, NEWTON EVERETT BIOTECH E-400 w/ rose hips, MUSHROOM SCIENCE lion's mane (Evidencia), NAC, Bacopa monnieri, LIFE EXTENSION inositol (evidencia), SOMESTA NEWTON EVERETT BIOTEC (BIOVEA), PURITAN'S PRIDE ginger root, PURITAN'S PRIDE' SAMe).

FIG. 2 illustrates additional components that can be included in particular embodiments, of a multi-component formulation shown in FIG. 1.

FIG. 3 shows one embodiment, of a blister packaging system for delivery of a multi-component formulation as described herein in conjunction with an active agent composition (e.g., tropisetron). As shown, the packaging system comprises a blister packaging card having bubble (blister) encapsulated tablets for administration at the times shown on the card.

FIG. 4 shows the use of the RxMap® perforated multiheat seal punch card packaging (MTS Medication technologies) for packaging a multi-component formulation as described herein and/or one or more pharmaceuticals or active agents (e.g., tropisetron). The RxMAP® packaging system is available in different sizes and formats. The card's inside cover provides the space to clearly label each prescription and associated instructions. In addition to these benefits, the perforated card allows the patient to take their medications with them in a smaller container. These individual blister packages are useful for “On-The-Go” patients as they can easily be carried in a pocket or a purse.

### DETAILED DESCRIPTION

In various embodiments, a new, non-stimulating mental enhancer for improving neurological function, cognitive ability, memory and mental acuity is provided as well as well as methods of using the same are provided. The combination of components, referred to herein as SYNAPTIK™, comprises a supplement system that raises the levels of factors in the brain that support brain health and wellness. SYNAPTIK™'s formulation (a combination of individual components) addresses deficiencies, particularly associated with the neurophysiological and structural changes in the brain that accompany aging as well as other brain disorders. The formulation(s) can be used to maintain or improve neurological health and/or function. In certain embodiments, the formulations can be used for treating neurodegenerative disorders, including, but not limited to diabetic neuropathy, ALS, Parkinson's disease Alzheimer's disease, age-related dementia, and precursor conditions in the Alzheimer's disease progression (e.g., MCI and various marker positive but otherwise asymptomatic conditions).

The formulations described herein are also contemplated, in part, for use in conjunction with various pharmaceuticals for the treatment and/or prophylaxis of Alzheimer's disease or other neurodegenerative conditions.

Many molecular targets have been implicated in the etiology of Alzheimer's disease. These include, for example, ApoE, alpha7 nAChR, APP, tau, vitamin D receptor (by SNP), MTHFR, estrogen receptor, GM-CSF receptor, and the like as well as molecules involved in inflammation, lipid transport, energy metabolism, and so forth. Without being bound by a particular theory regarding Alzheimer's etiology, it is believed that these seeming unrelated molecules and molecular targets mediate the etiology of Alzheimer's disease and other neurodegenerative pathologies. One mechanistic explanation is the possibility that these molecular targets function as dependence receptors.

It has been known for over half a century that cells depend for their survival on stimulation that is mediated by various receptors and sensors. For example, cells may require specific soluble trophic factors, cytokines, hormones, extracellular matrix interactions, cell-cell interactions, or electrical activity for survival. In each case, withdrawal of the stimulus leads to apoptosis. It has generally been assumed that this occurs through the loss of the associated positive survival signals, such as Akt phosphorylation.

While such survival signals are important, our data show that a complementary and novel form of signal transduction induces apoptosis and is activated by stimulus withdrawal. This “negative signal transduction” can be mediated by specific “dependence receptors” that induce apoptosis only in the absence of the required stimulus (e.g., when unbound by a trophic ligand). Thus, the expression of various dependence receptors creates states of dependence on their respective ligands.

Such receptors can regulate neurite retraction and cell death following trophic factor withdrawal (or anti-trophin interaction), and, conversely, they can mediate neurite extension, synaptic maintenance, and inhibition of programmed cell death (PCD) following trophic factor binding. These seemingly unrelated molecular targets (e.g., dependence receptors) have been implicated in Alzheimer's disease. In addition, seemingly unrelated effects of Aβ have been described, including, but are not limited to, inhibition of choline uptake, insulin signaling, NGF signaling, ACh neurotransmission, axonal transport, AMPA receptor recycling, reduced neural transmission, neurite retraction, caspase activation, PCD, etc. These effects are all linked by the process of plasticity (inhibition in some cases, activation at low concentrations).

Dependence receptors function as a system of integrating, analog-to-digital converters, sensing multiple biochemical concentrations (trophic factors, ECM, neurotransmitters, electrical activity, hormones, vitamins, etc.). The importance of each is based on receptor concentration and respective signaling (so, by analogy to synapses, different receptors are more or less contributory to the outcome).

As the brain ages and/or in neurodegenerative conditions, the system of dependence receptors may become progressively “unbalanced” leading to progressive neural dysfunction. Accordingly, to restore “balance” to this complex system of dependence receptors and improve or maintain neurological function a substantial number of dependence receptors (or classes of such receptor) should be administered their respective trophic ligand(s). Where there is cross-coverage between dependence receptors (or classes of depended receptors), e.g., via internal signals, then sufficient coverage can be afforded by a subset of trophic ligands. However, in general effective treatment of the dependence receptor imbalance is effectively addressed by administration of one or more of the multi-component formulations disclosed herein (e.g., as illustrated in Table 1 below).

The formulation(s) described herein are designed to address a substantial number, perhaps the majority, and desirably substantially all, (classes of) dependence receptors that contribute to neurological dysfunction associated with aging and/or other neurodegenerative pathologies. While other companies (e.g., DANNON®, THORNE®) purport to formulate nutrient-based solutions to counteract cognitive impairment and enhance normal function, such formulations typically only address a small portion, if any, of the depleted elements in brain function. Thus, because such formulations are not based on rectifying dependence receptor imbalance, at best, such formulations only partially and serendipitously address the imbalance e.g. Accordingly, it is believed that existing formulations in the art do not allow for maximum restoration of neurological function.

Particular embodiments, of the formulations described herein represent formulations that can comprehensively address the dependence receptor imbalance; thus, providing relief to all affected brain areas. Accordingly, it is believed that these only formulations that can fully enhance neurological function and physiology, cognitive function, memory, muscle movement control, etc., particularly in the context of a neurodegenerative pathology. In certain embodiments, omitting a portion of the multi-component formulation and/or one or more active agents may allow the pathogenesis to continue; thus, certain preferred formulations address most, if not, all of the known pathophysiological mechanisms in a network therapeutics fashion, and are believed to be materially and fundamentally distinct from all of the other currently marketed therapies.

By virtue of their design, the formulation(s) described herein are well suited to address the cognitive function decline in the elderly and in particular those with early or established neuropsychiatric disease, such as those with MCI or other pre-Alzheimer's conditions, in addition to Alzheimer's disease, Parkinson's disease, ALS, and other neurodegenerative conditions. Additionally, the formulations described herein can improve memory and mental skill of healthy individuals: professionals such as business executives, scientists, engineers, physicians, people generally on demanding assignments and even students, or simply those that want to maintain a high level mental acuity.

### Multi-Component Formulations.

Without being bound to a particular theory for Alzheimer's etiology, it is believed that at least part of the therapeutic effect of the multi-component formulations described herein relies on the fact that the formulation comprises sufficient components to provide ligands that activate a plurality, preferably a substantial number of dependence receptors in the brain. It is believed that such activation restores a healthy balance to this complex system of receptors and thereby helps “normalize” and thereby improve brain function. This is believed to be of value in the treatment of subjects identified with neurodegenerative pathologies (e.g., Alzheimer's disease, Parkinson's disease, and the like), in the treatment of precursors to such pathologies e.g., MCI, and in the treatment/prophylaxis of substantially asymptomatic individuals, or individuals where the only symptomology is a predilection disease indicated by, for example family history, markers, genetic screening, and the like.

The above mechanisms are not all-inclusive and many others may additionally be in operation in effecting the neural function that this treatment modality provides.

Table 1 illustrates certain preferred components of the formulations contemplated herein and the various components are listed by function. In certain embodiments, the formulation comprises: at least a first component comprising one or more vitamins selected from the group consisting of B vitamins, vitamin C, vitamin D, vitamin E, co-enzyme Q10, vitamin K, and folate; a second component comprising one or more elements or minerals selected from the group consisting of selenium, lithium, magnesium and molybdenum; a third component comprising one or more fatty acids (e.g., omega-3 fatty acids); and a fourth component comprising one or more amino acids (e.g., trimethylglycine, N-acetyl cysteine, S-adenosyl methionine, L-tryptophan, glutathione, and the like). Thus, in certain embodiments, the formulations comprise one or more of each of vitamin(s), element(s), fatty acid(s), and amino acids.

In certain embodiments, the formulation(s) further comprise a fifth component comprising one or more herbs (e.g., lion's main (Hericium), Bacopa monnieri, Ginkgo biloba, honokiol, magnolia extract, rosemary extract, ashwagandha, blueberry extract, billberry extract, ginger, he shou wu, rhodiola, reishi, saffron, daffodil, and the like). In certain embodiments, the formulation(s) further comprise a sixth component comprising one or more active agents selected from the group consisting of melatonin, pregnenolone, galangin, inpocetine, astaxanthine, and huperzine A. In various embodiments, the formulation further comprises a seventh component comprising a synthetic or natural phenol (e.g., a curcuminoid).

In certain embodiments, the formulation further comprises an eighth component comprising a lipid or phospholipid (e.g., phosphatidyl choline, choline, phosphatidyl serine, lipoic acid, and the like). In certain embodiments, the formulation further comprises a ninth component comprising a carbohydrate (e.g., inositol).

In certain embodiments, the fifth, sixth, seven eighth, and ninth components are arbitrarily numbered, for example, a multi-component formulation may comprise one or more of each of vitamin(s), element(s), fatty acid(s), and amino acids, and a fifth component that is a lipid or phospholipid, an herb, one or more active agents, a synthetic or natural phenol or a carbohydrate. In particular embodiments, the fifth, sixth, seven eighth, and ninth components are not arbitrarily numbered.

As used herein the term “vitamin” includes a naturally occurring vitamin, a vitamin precursor, a salt derivative of a vitamin, a vitamin ester, or a metabolite thereof, either in a natural or synthetic form. Examples of vitamins that can be included in the formulations described herein include, but are not limited to B vitamins, vitamin C, vitamin D, vitamin E, co-enzyme Q10, vitamin K, and folate. In certain embodiments, preferred B vitamins include vitamin B1, vitamin B2, vitamin B3, vitamin B5, vitamin B6, vitamin B7, vitamin B12, Bt (Carnitine), benfotiamine, and vitamin Bx (PABA), with vitamins B1, B5, B6, B12, and carnitine being preferred in particular. In certain embodiments, vitamins C, D, E, K, and folate are additionally preferred (see, e.g., Table 1). In certain embodiments, the vitamins comprise one or more vitamins selected from the group consisting of vitamin B1, vitamin C, vitamin E, vitamin K, and folate. In certain embodiments, the vitamins include all of vitamin B1, vitamin C, vitamin E, vitamin K, and folate. In certain embodiments, the vitamins include all of vitamin B1, vitamin B5, vitamin B6, vitamin B12, and vitamin B5 (Carnitine), vitamin C, vitamin E, vitamin K, and folate

As indicated above, in certain embodiments, the formulations contemplated herein additionally include one or more minerals or elements. As used herein, the term “mineral” refers to an element or chemical compound that is typically a naturally occurring solid chemical substance formed through biogeochemical processes, having characteristic chemical composition, highly ordered atomic structure, and specific physical properties. Minerals as used herein include isolated minerals, or salts thereof. Minerals or elements that may be used in the formulations described herein include, but are not limited to selenium, molybdenum, lithium, chromium, copper, iodine, iron, magnesium, manganese, phosphorus, potassium, and zinc with selenium, molybdenum, and lithium being preferred in particular embodiments, and selenium being preferred in certain embodiments (see, e.g., Table 1).

In addition to the vitamins and elements or minerals, the formulations described herein typically additionally contain one or more fatty acids, preferably omega-3 fatty acids. Omega-3 fatty acids (popularly referred to as ω-3 fatty acids or n-3 fatty acids) are fats commonly found in marine and plant oils. They are polyunsaturated fatty acids with a double bond (C=C) starting after the third carbon atom from the end of the carbon chain. The fatty acids have two ends—the acid (COOH) end and the methyl (CH₃) end. The location of the first double bond is counted from the methyl end, which is also known as the omega (ω) end or the n end. N-3 fatty acids may provide health benefits and are considered essential fatty acids, meaning that they cannot be synthesized by the human body but are important for normal metabolism. Suitable omega-3 fatty acids include, but are not limited to eicosapentaenoic acid, docosahexaenoic acid, and a-linolenic acid with eicosapentaenoic acid, and docosahexaenoic acid being particularly preferred in certain embodiments, (see, e.g., Table 1).

In various embodiments, the formulations additionally include one or more amino acids. Illustrative amino acids include, but are not limited to trimethylglycine, L-tryptophan, N-acetyl-cysteine, S-adenosyl methionine (SAMe), glutathione, and the like with trimethylglycine, N-acetyl-cysteine, and S-adenosyl methionine (SAMe) being particularly preferred in certain embodiments.

TABLE 1
Illustrative Synaptik ™ components listed by function.

| | | | Exemplary | | |
|---|---|---|---|---|---|
| | | | multi- | Exemplary multi- | |
| | | | component | component | Role/ |
| Class | Type | Subtype | formulation | formulation | Function(s) |
| vitamins | B | B1 | B1 | thiamine | block tau phosphorylation |
| | | B2 | | | |
| | | B3 | B3 | | |
| | | B5 | B5 | pantothenic acid | increase alertness |
| | | B6 | pyridoxine | pyridoxal-5- | reduce homocysteine |
| | | | | phosphate (P5P) | |
| | | B7 | | | |
| | | B12 | B12 | methyl cobalamin or | reduce homocysteine |
| | | | | hydroxycobalamin | |
| | | Bt (Carnitine) | L-carnitine | acetyl-L-camitine | increase NGF levels |
| | | benfotiamine | N/A | N/A | anti-oxidant |
| | | Bx (PABA) | N/A | N/A | folate precursor |
| | C | N/A | N/A | N/A | anti-oxidant |
| | D | N/A | D3 | N/A | bind Vitamin D receptor |
| | E | mixed | mixed | mixed tocopherols | anti-oxidant |
| | | tocopherols | tocopherols | & tocotrienols | |
| | Co-enzyme Q10 | N/A | N/A | N/A | anti-oxidant |
| | K | K2 | N/A | N/A | |
| | folate | N/A | methyl-folate | 5-methyl-tetra- | reduce homocysteine |
| | | | | hydrofolate | |
| elements | selenium | seleno- | | selenomethionine | anti-oxidant |
| | | methionine | | | |
| | molybdenum | N/A | N/A | N/A | anti-oxidant |
| | lithium | lithimn | orotate or | orotate | block tau |
| | | carbonate, | aspartate | | phosphorylation, inhibit |
| | | orotate, | | | neurodegeneration |
| | | chloride or | | | |
| | | aspartate | | | |
| Carbohydrates | inositol | N/A | N/A | N/A | bslock Abeta |
| | | | | | oligomerization |
| omega-3 | docosahex- | | | | synaptogenesis |
| fatty acids | aenoic Acid | | | | |
| | eicosapentaenoic | | | | synaptogenesis |
| | acid | | | | |
| lipids & | phosphatidyl | N/A | N/A | N/A | synaptogenesis |
| phospholipids | choline | | | | |
| | choline | Citicoline | CDP-choline | CDP choline | synaptogenesis |
| | phosphatidyl | N/A | N/A | N/A | synaptogenesis |
| | Serine | | | | |
| | lipoic Acid | N/A | Alpha-lipoic | N/A | synaptogenesis |
| | | | acid? | | |
| amino | trimethyl- | N/A | N/A | N/A | reduce homocysteine |
| acids - | glycine | | | | |
| derivatives - | L-tryptophan | N/A | N/A | N/A | |
| small | N-acetyl- | N/A | N/A | N/A | anti-oxidant: increase |
| peptides | cysteine (NAC) | | | | intracellular glutathione |
| | S-adenosyl | N/A | N/A | N/A | reduce homocysteine |
| | methionine | | | | (note SAMe is low in AD) |
| | (SAMe) | | | | |
| | glutathione | N/A | N/A | N/A | increase intracellular |
| | | | | | glutathione |
| natural | curcuminoids | curcumin, | in | curcumin bioperine | block Abeta |
| phenols | | turmeric | combination | | oligomerization; anti- |
| | | | with bioperine | | inflammatory; anti- |
| | | | | | oxidant |
| other | vinpocetine | N/A | N/A | N/A | anti-inflammatory |
| | astaxanthine | N/A | N/A | N/A | potent anti-oxidant |
| | pregnenolone | N/A | pregnenolone | pregnenolone | improve memory function |
| | | | acetate | acetate | |
| | huperzine A | N/A | N/A | N/A | AChE inhibitor; NMDA |
| | | | | | antagonist |
| | galangin | N/A | N/A | N/A | ASBI |
| herbs | lion's mane | N/A | N/A | N/A | increase NGF |
| | Bacopa | N/A | N/A | N/A | anti-oxidant; reduction of |
| | monnieri | | | | divalent metals |
| | Ginkgo biloba | N/A | ginkgo | ginkgo phytosome | multiple mechanisms, |
| | | | phytosome | (complex with PC) | including inhibition of |
| | | | (complex with | | thrombosis, inhibition of |
| | | | PC) | | norepinephrine reuptake, |
| | | | | | and other less well |
| | | | | | characterized |
| | honokiol | N/A | N/A | N/A | autophage activation; |
| | | | | | neuroprotective |
| | magnolia extract | N/A | N/A | N/A | |
| | rosemary extract | N/A | N/A | N/A | |
| | ashwagandha | N/A | N/A | N/A | anti-inflammatory; |
| | | | | | adaptogenic |
| | blueberry extract | N/A | blueberry leaf | N/A | anti-inflammatory |
| | | | extract | | |
| | billberry extract | N/A | N/A | N/A | anti-oxidant |
| | ginger | N/A | N/A | N/A | anti-inflammatory; better |
| | | | | | acid secretion |
| | he shou wu | N/A | N/A | N/A | |
| | rhodiola | N/A | N/A | N/A | monoamine oxidase |
| | | | | | inhibition |
| | reishi | N/A | N/A | N/A | |
| | saffron | N/A | N/A | N/A | |
| | daffodil | N/A | N/A | N/A | |

In various embodiments, the formulations additionally include one or more herbs. As used herein, the term “herb” refers to a fresh or dried part of a plant or a whole plant or an extract thereof, which comprises a biological activity (e.g., as identified in Table 1). Examples of herbs that can be used in the multicomponent formulations contemplated herein include, but are not limited to Allum sativum (garlic), black currant (Ribes nigra), bromlain, echinacea, ginseng (panax), ginseng (Siberian), hydrastasis, Medicago sativa (Alfalfa), passiflora, Ruscus aculeatus, St. John wort (Hypericum perforatum), Vaccinium myrtillus, lion's mane, Bacopa monnieri, Gingko biloba, honokiol, magnolia extract, rosemary extract, ashwagandha, blueberry extract, billberry extract, ginger, he shou wu, rhodiola, reishi, and saffron. In certain embodiments, the herb comprises one or more herbs selected from the group consisting of lion's mane, Bacopa monnieri, Gingko biloba, honokiol, magnolia extract, rosemary extract, ashwagandha, blueberry extract, billberry extract, ginger, he shou wu, rhodiola, reishi, and saffron. In certain embodiments, the herbs comprise one or more herbs selected from the group consisting of lion's mane (Hericium erinaceous), Bacopa monnieri, Gingko biloba, honokiol, and ginger. In certain embodiments, the herb comprises at least lion's mane (Hericium erinaceous), Bacopa monnieri, Gingko biloba, honokiol, and ginger (e.g., as identified in Table 1).

A wide range of methods is known in the art for the production of therapeutics from herbs. For example, herbs may be subjected to a polar (e.g., aqueous) solvent extraction. The aqueous extract may then be filtered if necessary to remove large particles, and subsequently dried (e.g., by exposure to warm dry air (e.g., 65° C.) for a length of time such as three days to one week) to a powder. Alternatively, it is possible to use dry herbs directly by grinding to a powder. A number of herbs, herbal tinctures and herbal extracts are available from commercial suppliers.

In various embodiments, embodiments, the multi-component formulations contemplated herein further include one or more active agents selected from the group consisting of melatonin, pregnenolone, and galangin.

The multi-component formulations contemplated herein can also further include a naturally occurring or synthetic phenol. In certain embodiments, the phenol comprises a curcuminoid and/or turmeric.

In certain embodiments, the multi-component formulations include a lipid or phospholipid. Illustrative lipids or phospholipids include, but are not limited to phosphatidyl choline, choline, phosphatidyl serine, and lipoic acid. In certain embodiments, the lipid or phospholipid comprises choline. A carbohydrate can be present in the multi-component formulations and when present, in some embodiments, comprises inositol.

In certain embodiments, the multi-component formulation comprises at least four agents selected from the group consisting of vitamin B1, vitamin B5, vitamin B6, vitamin B12, carnitine, vitamin C, vitamin D, vitamin E, vitamin K, folate, selenium, lithium, docosahexaenoic acid, eicosapentaenoic acid, choline, trimethylglycine, L-tryptophan, N-acetyl-cysteine, S-adenosyl methionine (SAMe), melatonin, pregnenolone, galangin, lion's mane (Hericium erinaceous), Bacopa monnieri, Ginkgo biloba, honokiol, and ginger, wherein the four different agents comprise at least four different components. In certain embodiments, the formulation comprises at least five different agents selected from this group and the five different agents comprise at least five different components. In certain embodiments, the formulation comprises at least six different agents selected from this group and the six different agents comprise at least six different components. In certain embodiments, the formulation comprises at least seven different agents selected from this group the seven different agents comprise at least seven different components. In certain embodiments, the multi-component formulation comprises at least eight different agents selected from this group the eight different agents comprise at least eight different components.

In certain embodiments, the multi-component formulation comprises a first component comprising vitamin B1, and/or vitamin B5, and/or vitamin B6, and/or vitamin B12, and/or carnitine, and/or vitamin C, and/or vitamin E, and/or vitamin K, and/or folate, a second component comprising selenium and/or lithium; a third component comprising an omega-3 fatty acid; a fourth component comprising trimethylglycine, and/or N-acetyl cysteine, and/or S-adenosyl methionine; a fifth component comprising lion's mane, and/or Bacopa monnieri, and/or Ginkgo biloba, and/or honokiol; and a sixth component wherein said sixth component comprises melatonin, and/or pregnenolone, and/or galangin. In certain embodiments, the first component comprises vitamin B1, vitamin B5, vitamin B6, vitamin B12, carnitine, vitamin C, vitamin E, vitamin K, and folate; the second component comprises selenium and/or lithium; the third component comprises docosahexaenoic acid, and/or eisopentanoic acid; the fourth component comprises trimethylglycine, N-acetyl cysteine, and S-adenosyl methionine; the fifth component comprises lion's mane, Bacopa monnieri, Ginkgo biloba, and honokiol; and the sixth component comprises melatonin, pregnenolone, and galangin. In certain embodiments, the formulation further comprises said seventh component, where the seventh component comprises a curcuminoid. In certain embodiments, the formulation further comprises said eighth component, where said eighth component comprises a lipid or phospholipid (e.g., choline). In certain embodiments, the formulation further comprises a ninth component, wherein the ninth component comprises inositol.

In various embodiments, various components comprising the multi-component formulation, when present, are present in the ranges shown in Table 2. In certain embodiments, of the multi-component formulation, B1, when present, comprises at least about 100 mg; vitamin B5, when present, comprises at least 25 mg; vitamin B6, when present, comprises at least 5 mg; vitamin B12, when present, comprises at least about 0.1 mg; vitamin C, when present, comprises at least about 2,000 mg, vitamin D, when present, comprises at least about 1000 IU; vitamin E, when present, comprises at least about 50 mg; vitamin K, when present, comprises at least about 10 mg; folate, when present, comprises at least about 0.2; selenium, when present, comprises at least about 25 μg; lithium, when present, comprises at least about 1 mg; inositol, when present, comprises at least about 0.25 mg; docosahexaenoic acid, when present, comprises at least about 0.25 g; eicosapentanoic acid, when present, comprises at least about 0.25 g; choline, when present, comprises at least about 0.5 g; trimethylglycine, when present, comprises at least about 120 mg; N-acetyl-cysteine, when present, comprises at least about 200 mg; S-adenosyl methionine, when present, comprises at least about 100 mg; a curcuminoid, when present, comprises at least about 500 mg; melatonin, when present, comprises at least about 1 mg; pregnenolone, when present, comprises at least about 2 mg; galangin, when present, comprises at least about 200 mg; lion's mane, when present, comprises at least about 250 mg; Bacopa monnieri, when present, comprises at least about 50 mg; Ginkgo biloba, when present, comprises at least about 20 mg;honokiol, when present, comprises at least about 200-1000 mg; and Ginger, when present, comprises at least about 100 mg. In certain embodiments, of the multi-component formulation, vitamin B1, when present, ranges from about 100 to about 750 mg; vitamin B5, when present, ranges from about 25 to about 150 mg; vitamin B6, when present, ranges from about 5 to about 50 mg; vitamin B12, when present, ranges from about 0.1 mg to about 3 mg; acetyl-L-carnitine (ALCAR), when present, ranges from about 250-2000 mg; and vitamin C, when present, ranges from about 100-1000 mg; vitamin D, when present, ranges from about 1000 IU to about 5000 IU; vitamin E, when present, ranges from about 50 mg to about 1500 mg; vitamin K, when present, ranges from about 10 mg to about 200 mg; folate, when present, ranges from about 0.2 mg to about 1.5 mg; selenium, when present, ranges from about 25 μg to about 500 μg; lithium, when present, ranges from about 1 mg to about 20 mg; inositol, when present, ranges from about 500 mg to about 4000 mg; docosahexaenoic acid, when present, ranges from about 0.25 g to about 1.5 g; eicosapentaenoic acid, when present, ranges from about 0.25 g to about 1.5 g; choline, when present, ranges from about 0.5 g to about 3 g; trimethylglycine, when present, ranges from about 120 mg to about 1000 mg; N-acetyl-cysteine, when present, ranges from about 200 mg to about 1000 mg; S-adenosyl methionine, when present, ranges from about 100 mg to about 600 mg; a curcuminoid, when present, ranges from about 500 mg to about 4000 mg; melatonin, when present, ranges from about 1 mg to about 4 mg; pregnenolone, when present, ranges from about 2 mg to about 5 mg; galangin, when present, ranges from about 200 mg to about 1000 mg; lion's mane, when present, ranges from about 250 mg to about 2000 mg; Bacopa monnieri, when present, ranges from about 50 mg to about 600 mg; Ginkgo biloba, when present, ranges from about 20 mg to about 200 mg; honokiol, when present, ranges from about 1 mg (thus, for a 2% extract, 50 mg of the 2% extract) to about 25 mg (i.e., 1.25 g of 2% extract); and ginger, when present, ranges from about 100 mg to about 1000 mg. In certain embodiments, vitamin B1 is present and ranges from about 2 to about 500 mg; vitamin B5 is present and ranges from about 25 to about 350 mg; vitamin B6 is present and ranges from about 5 to about 50 mg; vitamin B12 is present and ranges from about 0.1 mg to about 3 mg; acetyl-L-carnitine (ALCAR) is present and ranges from about 250-2000 mg; vitamin C is present and ranges from about 100-1000 mg; vitamin D is present and ranges from about 1000 IU to about 5000 IU; vitamin E is present and ranges from about 50 mg to about 1500 mg; vitamin K is present and ranges from about 10 mg to about 200 mg; folate is present and ranges from about 0.2 mg to about 1.5 mg; selenium is present and ranges from about 25 μg to about 500 μg; lithium is present and ranges from about 1 mg to about 20 mg; inositol is present and ranges from about 500 mg to about 4000 mg; docosahexaenoic acid is present and ranges from about 0.25 g to about 1.5 g; eicosapentanoic acid is present and ranges from about 0.25 g to about 1.5 g; choline is present and ranges from about 0.5 g to about 3 g; trimethylglycine is present and ranges from about 120 mg to about 1000 mg; N-acetyl-cysteine is present and ranges from about 200 mg to about 1000 mg; S-adenosyl methionine is present and ranges from about 100 mg to about 600 mg; a curcuminoid is present and ranges from about 500 mg to about 4000 mg; pregnenolone is present and ranges from about 2 mg to about 25 mg; galangin is present and ranges from about 200 mg to about 4000 mg; lion's mane is present and ranges from about 250 mg to about 2000 mg; Bacopa monnieri is present and ranges from about 50 mg to about 600 mg; Ginkgo biloba is present and ranges from about 20 mg to about 200 mg; Honokiol is present and ranges from about 1 mg to about 25 mg; and ginger is present and ranges from about 100 mg to about 1000 mg.

TABLE 2
Illustrative dosage ranges and illustrative dose levels for the various
elements that can comprise a multi-component formulation for the treatment and/or
prophylaxis of a neurodegenerative disorder. Where no dosage is indicated, in certain
embodiments, the subject can be administered up to the maximum daily recommended dose
for that component.

| | | | Exemplary | | Exemplary | | Exemplary | | Exemplary | Exemplary |
|---|---|---|---|---|---|---|---|---|---|---|
| | | | Daily Dose | | Daily Dose | | Daily Dose | | Admin. | Admin. |
| Class | Type | Subtype | Range | | Range | | Range | | Schedule | Schedule |
| Vitamins | B | B1 | 2.5-750 | mg | 125-500 | mg | 250 | mg | qd or bid | bid |
| | | B2 | | | | | | | | |
| | | B3 | | | | | | | | |
| | | B5 | 25-150 | mg | 50-100 | mg | 75 | mg | qd or bid | bid |
| | | B6 | 5-50 | mg | 10-20 | mg | 10 | mg | qd or bid | bid |
| | | Pyridoxine | | | | | | | | |
| | | B7 | | | | | | | | |
| | | B12 | 0.1-3 | mg | 0.5-2 | mg | 0.5-1 | mg | qd or bid | bid |
| | | Bt (Carnitine) | | | | | | | | |
| | | L-Carnitine | | | | | | | | |
| | | Benfotiamine | 10-400 | mcg | 20-300 | mcg | 150 | mcg | qd or bid | bid |
| | | Bx (PABA) | 10-400 | mg | 100-300 | mg | 150 | mg | qd or bd | bid |
| | C | N/A | 5000-4000 | mg | 500-3000 | mg | 1000 | mg | qd or bid | qd or bid |
| | D | D3 | 1000-4000 | IU | 2000-4000 | IU | 2000 | IU | qd | bid |
| | | | | | 1000-2000 | IU | 1000 | IU | | |
| | E | Mixed | 50-1500 | mg | 300-1000 | mg | 500 | mg | bid | bid |
| | | tocopherols | | | | | 1000 | mg | qd or bid | |
| | Co-enzyme | N/A | 10-300 | mg | 50-200 | mg | 300 | mg | qd or bid | bid |
| | Q10 | | | | | | | | | |
| | K | K2 | 10-200 | mg | 25-150 | mg | 50-100 | mg | bid or qd | bid |
| | Folate | Methyl folate | 0.2-1.5 | | 0.6-1.0 | | 0.8 | mg | qd | bid |
| | | | | | 0.2-0.6 | | 0.4 | mg bid | bid | |
| Elements | Selenium | Selenomethionine | 25-500 | mcg | 100-200 | mcg | 100 | ncg | bid | bid |
| | Molybdenum | N/a | 10-50 | mcg | 20-40 | mcg | 25 | mcg | | |
| | Lithium | Lithium | 1-20 | mg | 5-10 | mg | 5 | mg | bid | bid |
| | | carbonate, | | | | | | | | |
| | | orotate, | | | | | | | | |
| | | chloride or | | | | | | | | |
| | | aspartate | | | | | | | | |
| Carbohydrates | Inositol | N/A | 0.25-3 | g | 0.75-2.5 | g | 1 | g | bid | bid |
| Omega-3 | Docosahexaenoic | | 0.25-1.5 | g | 0.5-1 | g | 1 | g | qd | bid |
| Fatty Acids | Acid | | | | | | 0.5 | g | bid | |
| | Eicosapentaenoic | | 0.25-1.5 | g | 0.5-1 | g | 1 | g | qd | bid |
| | Acid | | | | | | 0.5 | g | bid | |
| Lipids & | Phosphatidyl | N/A | 50-500 | mg | 100-200 | mg | 150 | mg | qd or bid | bid |
| Phospholipids | choline | | | | | | | | | |
| | Choline | Citicholine | 0.5-3 | g | 1.5-3 | g | 2 | g | qd | bid |
| | | CDP Choline | | | 0.5-1 | g | 750 | mg | bid | |
| | Phosphatidyl | N/A | 10-1000 | mg | 100-1000 | mg | 500 | mg | qd or bid | bid |
| | Serine | | | | | | | | | |
| | Lipoic Acid | N/A | 10-500 | mg | 50-200 | mg | 100 | mg | qd or bid | bid |
| Amino | Trimethylglycine | N/A | 120-1000 | mg | 250-500 | | 250 | mg | bid | bid |
| Acid | | | | | | | 500 | mg | qd | |
| Derivatives | L-Tryptophan | N/A | 250-1000 | mg | 300-700 | mg | 500 | mg | qd | |
| and Small | N-Acetyl-Cysteine | N/A | 200-1000 | mg | 400-800 | mg | 500 | mg | qd or bid | bid |
| Peptides | S-Adenosyl | N/A | 100-600 | mg | 200-400 | mg | 200 | mg | qd or bid | bid |
| | Methionine | | | | | | | | | |
| | (SAMe) | | | | | | | | | |
| | Glutathione | N/A | 50-300 | mg | 100-200 | mg | 150 | mg | qd or bid | bid |
| Natural | Curcuminoids | Curcumin, | 500-4000 | mg | 1000-2000 | mg | 1000 | mg | qd or bid | bid |
| Phenols | | turmeric | | | | | | | | |
| | | Optionally in | | | | | | | | |
| | | combination | | | | | | | | |
| | | with bioperine | | | | | | | | |
| Other | Vinpocetine | N/A | 1-20 | mg | 5-15 | mg | 10 | mg | qd or bid | bid |
| | Melatonin | N/A | 1-4 | mg | 1-2 | mg | 1 | mg | qd bedtime | qd bedtime |
| | Astaxanthin | N/A | 2-5 | mg | 3-4 | mg | 4 | mg | qd or bid | qd |
| | Pregnenolone | Pregnenolone | 2-20 | mg | 5-10 | mg | 5 | mg | bid or qd | bid |
| | | acetate | | | | | | | | |
| | Huperzine A | N/A | 10-100 | mg | 25-50 | mg | 25 | mg | bid or qd | ?? |
| | Galangin | N/A | 200-1000 | mg | 400-1000 | mg | 500 | | bid or qd | bid |
| Herbs | Lion's Mane | N/A | 250-2000 | mg | 500-1000 | mg | 500 | mg | qd or bid | bid |
| | (Hericium | | | | | | | | | |
| | Erinaceus) | | | | | | | | | |
| | Bacopa monnieri | N/A | 50-600 | mg | 100-300 | mg | 200 | mg | qd or bid | bid |
| | Ginkgo biloba | Ginkgo | 20-200 | mg | 60-120 | mg | 60 | mg | qd or bid | bid |
| | | Phytosome | | | | | | | | |
| | | (complex with PC) | | | | | | | | |
| | Honokiol | N/A | | | | | | | | |
| | Magnolia | N/A | 20-300 | mg | 50-150 | mg | 100 | mg | qd or bid | bid |
| | extract | | | | | | | | | |
| | Rosemary | N/A | 20-300 | mg | 50-150 | mg | 100 | mg | qd or bid | bid |
| | extract | | | | | | | | | |
| | Ashwagandha | N/A | 100-500 | mg | 200-400 | mg | 300 | mg | qd or bid | bid |
| | Blueberry | Blueberry leaf | 100-500 | mg | 200-400 | mg | 300 | mg | qd or bid | bid |
| | extract | extract | | | | | | | | |
| | Billberry | N/A | 100-500 | mg | 200-400 | mg | 300 | mg | qd or bid | bid |
| | extract | | | | | | | | | |
| | Ginger | N/A | 100-1000 | mg | 300-700 | mg | 500 | mg | qd or bid | bid |
| | He Shou Wu | N/A | 500-4000 | mg | 750-2000 | mg | 1000 | mg | qd or bid | bid |
| | Rhodiola | N/A | 200-4000 | mg | 750-2000 | mg | 1000 | mg | qd or bid | bid |
| | Reishi | N/A | 200-4000 | mg | 750-2000 | mg | 1000 | mg | qd or bid | bid |
| | Saffron | N/A | 200-4000 | mg | 750-2000 | mg | 1000 | mg | qd or bid | bid |
| | Daffodil | N/A | 200-4000 | mg | 750-2000 | mg | 1000 | mg | qd or bid | bid |

The foregoing combinations and dosages are illustrative and not necessarily limiting. In various embodiments, other combinations of the components and ranges shown in Table 2 comprising at agents from at least 5, preferably at least 6, more preferably at least seven, and most preferably at least 8 different classes shown Table 2 will be present in a multi-component formulation.

Typically, the multi-component formulations will be administered in an amount effective to achieve the intended purpose. In various embodiments, an effective amount is an amount sufficient to improve at least one symptom associated with a neurological disorder, improve neurological function, improve cognition, or one or more markers of a neurological disease, or to enhance the efficacy of one or more pharmaceuticals administered for the treatment or prophylaxis of a neurodegenerative pathology. In certain embodiments, an effective amount is an amount sufficient alone, or in combination with a therapeutic agent to inhibit or prevent the onset, and/or to slow the progression, and/or to lessen the severity of a neurodegenerative pathology. Exemplary effective doses are provided in Table 2.

In light of the detailed disclosure provided herein, one having ordinary skill in the art, would be able to determine a therapeutically effective amount a multi-component formulation disclosed herein.

Toxicity and therapeutic efficacy of the constituents of the multi-component formulation(s) described herein can be determined/verified by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The route of administration and dosage can be chosen by the individual physician in view of the patient's condition. (See e.g., Fingl, et al., 1975, in “The Pharmacological Basis of Therapeutics”, Ch. 1 p.1).

The amount of a composition to be administered will, of course, be dependent on the subject being treated, the severity of the affliction, the manner of administration, the judgment of the prescribing physician, etc.

### Combination Therapies

In certain embodiments, multi-component formulations described herein can be used in combination with other therapeutic agents or approaches used to treat or prevent neurodegenerative pathologies (e.g., early stage Alzheimer's disease, late stage Alzheimer's disease, age-related dementia, Parkinson's disease, Huntington's disease, multiple sclerosis, amyotrophic lateral sclerosis (ALS or Lou Gehrig's Disease), prion diseases, Creutzfeldt-Jakob disease, Lewy body disease, Friedreich's ataxia, stroke, genetic brain disorders), or precursors of such conditions (e.g., pre-Alzheimer's, mild cognitive impairment (MCI), and the like). Without being bound to a particular theory, it is believed that by “normalizing” the neurophysiology of the brain, neurological function is improved and the multi-component formulations described herein can thereby enhance the efficacy of other therapeutics used in the treatment of neurodegenerative pathologies, and/or neurodegeneration simply associated with aging. The formulations disclosed herein also improve cognitive function in individuals without neurodegeneration, as well as those in the pre-symptomatic phases.

Accordingly, in certain embodiments, the use of the multi-component formulations described herein in conjunction with one or more additional therapeutic agents is contemplated. In certain embodiments, such therapeutic agents include, but are not limited to disulfiram and/or analogues thereof, honokiol and/or analogues thereof, tropisetron and/or analogues thereof, nimetazepam and/or analogues thereof (see, e.g., U.S. Ser. No. 13/213,960 (U.S. Patent Publication No: US-2012-0071468-A1), and PCT/US2011/048472 (PCT Publication No: WO 2012/024616) which are incorporated herein by reference for the compounds described therein), tropinol-esters and/or related esters and/or analogues thereof (see, e.g., U.S. Ser. No. 61/514,381, which is incorporated herein by reference for the compounds described herein), TrkA kinase inhibitors (e.g., ADDN-1351) and/or analogues thereof (see, e.g., U.S. Ser. No. 61/525,076, which is incorporated herein by reference for the compounds described therein), as well as D2 receptor agonists and alpha1-adrenergic receptor antagonists.

In certain illustrative embodiments, the multi-component formulations are used in conjunction with tropisetron (or tropisetron analogues, e.g., as described in U.S. Ser. No. 13/213,960 (US-2012-0071468-A1), and PCT/US2011/048472 (WO 2012/024616) which are incorporated herein by reference for the compounds (e.g., tropisetron and analogs thereof) listed therein.

In certain illustrative embodiments, the multi-component formulations are used in conjunction with tropinol esters (e.g., tropinol esters and related esters as described in U.S. Ser. No. 61/514,381, which is incorporated herein by reference for the compounds (e.g., tropinol esters and related esters) described therein).

The multi-component formulations described herein can also be used in conjunction with other drugs such as acetylcholinesterase inhibitors (including without limitation, e.g., (−)-phenserine enantiomer, tacrine, ipidacrine, galantamine, donepezil, icopezil, zanapezil, rivastigmine, huperzine A, phenserine, physostigmine, neostigmine, pyridostigmine, ambenonium, demarcarium, edrophonium, ladostigil and ungeremine); NMDA receptor antagonists (including without limitation e.g., memantine); muscarinic receptor agonists (including without limitation, e.g., talsaclidine, AF-102B, AF-267B (NGX-267)); alpha4 nicotinic receptor agonist nicotinic receptor agonists (including without limitation, e.g., ispronicline (AZD-3480)); alpha7 nicotinic receptor agonist with 5HT-3 antagonist activity (including without limitation e.g., tropisetron); beta-secretase (BACE-1)inhibitors (including without limitation e.g., thiazolidinediones, including rosiglitazone and pioglitazone or direct BACE-1 inhibitors belonging to statins, the hydroxyethylenes, the hydroxyethylamines, the cyclic ureas, or the aminohydantoin class of inhibitors); gamma-secretase inhibitors (including without limitation, e.g., semagacestat (LY-450139), MK-0752, E-2012, BMS-708163, PF-3084014, begacestat (GSI-953), and NIC5-15); inhibitors of Aβ aggregation (including without limitation, e.g., clioquinol (PBT1), PBT2, tramiprosate (homotaurine), scyllo-inositol (a.k.a., scyllo-cyclohexanehexol, AZD-103 and ELND-005), passive immunotherapy with Aβ fragments (including without limitations e.g., Bapineuzemab), GSK-3 kinase inhibitors (including without limitations e.g., Tideglusib); Receptor for Advanced Glycation Endproducts (RAGE) inhibitors (including without limitation e.g., PF 04494700); 5HT-4 agonist (including but without limitation e.g. PRX03140); 5HT-6 antagonist (including but without limitation e.g. SB742457); glial derived activity dependent neuroprotective protein (NAP) fragment (including but without limitation e.g., AL-108); PKC modulators (including but without limitation e.g., Byrostatin-1) and epigallocatechin-3-gallate (EGCG)); anti-inflammatory agents such as cyclooxygenase II inhibitors; anti-oxidants such as vitamin E and ginkolides; immunological approaches, such as, for example, immunization with Aβ peptide or administration of anti-Aβ peptide antibodies; statins; and direct or indirect neurotrophic agents such as Cerebrolysin™, AIT-082 (Emilieu (2000) Arch. Neurol. 57: 454), Netrin (Luorenco (2009) Cell Death Differ 16: 655-663), netrin mimetics, NGF, NGF mimetics, BDNF, BDNF mimetics, agents that promote neurogenesis e.g., stem cells, and other neurotrophic agents. Further, pharmacologic agents useful in combination with the multi-component formulations described herein are described, e.g., in Mangialasche et al. (2010) Lancet Neurol., 9: 702-716.

### Methods of Use

The methods described herein are based, in part, on the surprising discovery that the multi-component formulations described herein represent formulations that can comprehensively address the dependence receptor imbalance, and thus, it is believed, provide relief to substantially all affected brain areas. It is believed the formulations described herein can fully enhance neurological function and physiology, cognitive function, memory, muscle movement control, etc., particularly in the context of a neurodegenerative pathology.

In one embodiment, the result of restoring dependence receptor imbalance is promoting processing of amyloid beta (A4) precursor protein (“APP”) by the nonamyloidogenic (“anti-AD”) pathway and reducing or inhibiting processing of APP by the amyloidogenic (“pro-AD”) pathway. This is believed to result in reduced production of Aβ, which may be deposited as amyloid plaques in the brain, and the other pro-amyloidogenic fragments known to result in neurotoxicity.

In a particular embodiment, the multi-component formulations described herein can be used to mitigate or ameliorate in a mammal one or more symptoms associated with mild cognitive impairment (MCI), particular MCI associated with amyloid deposits in the brain.

In certain embodiments the multi-component formulations described herein can be used (alone or in combination with other active agents, e.g., as described herein) in a method of preventing or delaying the onset of a pre-Alzheimer's condition and/or cognitive dysfunction, and/or ameliorating one or more symptoms of a pre-Alzheimer's condition and/or cognitive dysfunction, or preventing or delaying the progression of a pre-Alzheimer's condition or cognitive dysfunction to Alzheimer's disease.

Additionally, in certain embodiments, these formulations can address the need to improve memory in healthy individuals that would benefit from a boost of their memory and mental skill, e.g., professionals such as business executives, scientists, people generally on demanding assignments and even students, or simply those that want to maintain a high level of mental acuity.

Accordingly, in various embodiments, methods are provided for the treatment and/or prevention, and/or improvement of at least one symptom associated with a neurological disorder or neurodegenerative disease, e.g., diseases characterized by an amyloidogenic process (e.g., MCI or the progression of MCI and/or other pre-Alzheimer's condition to Alzheimer's disease), or improvement of neurological function, e.g., cognition, memory, mental acuity, and the like.

In particular embodiments methods, are provided for improving at least one symptom associated with a neurological disorder or disease. In certain embodiments, cognition, memory, and/or mental acuity are improved. In certain embodiments, improved neurological function in a treated subject is evidence by reducing of one or more markers that are characteristic of the pathology or disease (e.g., of total-Tau (tTau), phospho-Tau (pTau), APPneo, soluble Aβ40, pTau/Ap42 ratio and tTau/Ap42 ratio, and/or an increase in the CSF of levels of one or more components selected from the group consisting of Aβ42/Aβ40 ratio, Aβ42/Aβ38 ratio, sAPPα, βAPPα/βAPPβ ratio, βAPPα/Aβ40 ratio, βAPPα/Aβ42 ratio, etc.) and/or reduction, stabilization or reversal of one or more diagnostic criteria (e.g., clinical dementia rating (CDR)).

In various embodiments, a subject is administered a multi-component formulation described herein alone, or in conjunction with one or more active agents (e.g., pharmaceuticals) as disclosed elsewhere herein.

In certain embodiments the methods involve administration of a multi-component formulation described therein, optionally in conjunction with one or more active agents (e.g., tropisetron, disulfiram, honokiol, and/or nimetazepam, tropinol esters and/or related esters) and/or an analog thereof for the prevention and/or treatment of diseases characterized by amyloid deposits in the brain, particularly MCI or the progression of MCI, or other pre-Alzheimer's condition to early stage Alzheimer's disease. In certain embodiments the multi-component formulations can be used alone or in conjunction with other active agents to ameliorate one or more symptoms of Alzheimer's disease as described herein.

Prophylaxis

In certain embodiments active agent(s) (e.g., tropinol esters and related esters, analogues, derivatives, or prodrugs thereof) are utilized in various prophylactic contexts. Thus, for example, in certain embodiments, the active agent(s) (e.g., tropinol esters) can be used to prevent or delay the onset of a pre-Alzheimer's cognitive dysfunction, and/or to ameliorate one more symptoms of a pre-Alzheimer's condition and/or cognitive dysfunction, and/or to prevent or delay the progression of a pre-Alzheimer's condition and/or cognitive dysfunction to Alzheimer's disease.

Accordingly in certain embodiments, the prophylactic methods described herein are contemplated for subjects identified as “at risk” and/or as having evidence of early Alzheimer's Disease (AD) pathological changes, but who do not meet clinical criteria for MCI or dementia. Without being bound to a particular theory, it is believed that even this “preclinical” stage of the disease represents a continuum from completely asymptomatic individuals with biomarker evidence suggestive of AD-pathophysiological process(es) (abbreviated as AD-P, see, e.g., Sperling et al. (2011) Alzheimer's & Dementia, 1-13) at risk for progression to AD dementia to biomarker-positive individuals who are already demonstrating very subtle decline but not yet meeting standardized criteria for MCI (see, e.g., Albert et al. (2011) Alzheimer's and Dementia, 1-10 (doi:10.1016/j.jalz.2011.03.008).

This latter group of individuals might be classified as “not normal, not MCI” but can be designated “pre-symptomatic” or “pre-clinical or “asymptomatic” or “premanifest”). In various embodiments this continuum of pre-symptomatic AD can also encompass (1) individuals who carry one or more apolipoprotein E (APOE) ε4 alleles who are known or believed to have an increased risk of developing AD dementia, at the point they are AD-P biomarker-positive, and (2) carriers of autosomal dominant mutations, who are in the presymptomatic biomarker-positive stage of their illness, and who will almost certainly manifest clinical symptoms and progress to dementia.

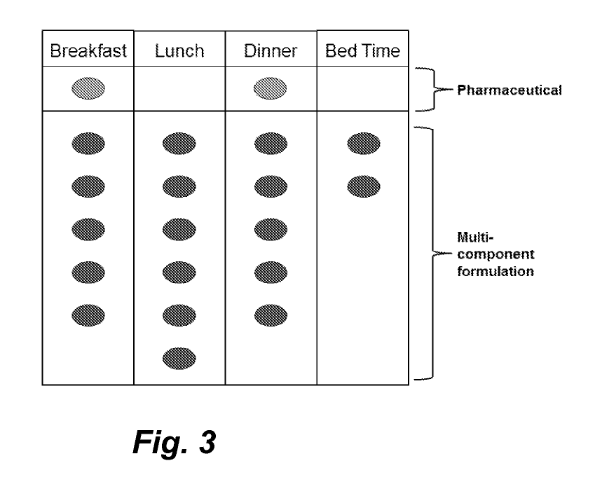

A biomarker model has been proposed in which the most widely validated biomarkers of AD-P become abnormal and likewise reach a ceiling in an ordered manner (see, e.g., Jack et al. (2010) Lancet Neurol., 9: 119-128.). This biomarker model parallels proposed pathophysiological sequence of (pre-AD/AD), and is relevant to tracking the preclinical (asymptomatic) stages of AD (see, e.g., FIG. 3 in Sperling et al. (2011) Alzheimer's & Dementia, 1-13). Biomarkers of brain amyloidosis include, but are not limited to reductions in CSF Aβ₄₂ and increased amyloid tracer retention on positron emission tomography (PET) imaging. Elevated CSF tau is not specific to AD and is thought to be a biomarker of neuronal injury. Decreased fluorodeoxyglucose 18F (FDG) uptake on PET with a temporoparietal pattern of hypometabolism is a biomarker of AD-related synaptic dysfunction. Brain atrophy on structural magnetic resonance imaging (MRI) in a characteristic pattern involving the medial temporal lobes, paralimbic and temporoparietal cortices is a biomarker of AD-related neurodegeneration. Other markers include, but are not limited to volumetric MRI, FDG-PET, or plasma biomarkers (see, e.g., Vemuri et al. (2009) Neurology, 73: 294-301; Yaffe et al. (2011) JAMA 305: 261-266).

In certain embodiments the subjects suitable for the prophylactic methods contemplated herein include, but are not limited to subject characterized as having asymptomatic cerebral amyloidosis. In various embodiments these individuals have biomarker evidence of Aβ accumulation with elevated tracer retention on PET amyloid imaging and/or low Aβ42 in CSF assay, but typically no detectable evidence of additional brain alterations suggestive of neurodegeneration or subtle cognitive and/or behavioral symptomatology.

It is noted that currently available CSF and PET imaging biomarkers of Aβ primarily provide evidence of amyloid accumulation and deposition of fibrillar forms of amyloid. Data suggest that soluble or oligomeric forms of Aβ are likely in equilibrium with plaques, which may serve as reservoirs. In certain embodiments it is contemplated that there is an identifiable preplaque stage in which only soluble forms of Aβ are present. In certain embodiments it is contemplated that oligomeric forms of amyloid may be critical in the pathological cascade, and provide useful markers. In addition, early synaptic changes may be present before evidence of amyloid accumulation.

In certain embodiments the subjects suitable for the prophylactic methods contemplated herein include, but are not limited to, subjects characterized as amyloid positive with evidence of synaptic dysfunction and/or early neurodegeneration. In various embodiments these subjects have evidence of amyloid positivity and presence of one or more markers of “downstream” AD-P-related neuronal injury. Illustrative, but non-limiting markers of neuronal injury include, but are not limited to (1) elevated CSF tau or phospho-tau, (2) hypometabolism in an AD-like pattern (i.e., posterior cingulate, precuneus, and/or temporoparietal cortices) on FDG-PET, and (3) cortical thinning/gray matter loss in a specific anatomic distribution (i.e., lateral and medial parietal, posterior cingulate, and lateral temporal cortices) and/or hippocampal atrophy on volumetric MRI. Other markers include, but are not limited to fMRI measures of default network connectivity. In certain embodiments early synaptic dysfunction, as assessed by functional imaging techniques such as FDG-PET and fMRI, can be detectable before volumetric loss. Without being bound to a particular theory, it is believed that amyloid-positive individuals with evidence of early neurodegeneration may be farther down the trajectory (i.e., in later stages of preclinical (asymptomatic) AD).

In certain embodiments the subjects suitable for the prophylactic methods contemplated herein include, but are not limited to, subjects characterized as amyloid positive with evidence of neurodegeneration and subtle cognitive decline. Without being bound to a particular theory, it is believed that those individuals with biomarker evidence of amyloid accumulation, early neurodegeneration, and evidence of subtle cognitive decline are in the last stage of preclinical (asymptomatic) AD, and are approaching the border zone with clinical criteria for mild cognitive impairment (MCI). These individuals may demonstrate evidence of decline from their own baseline (particularly if proxies of cognitive reserve are taken into consideration), even if they still perform within the “normal” range on standard cognitive measures. Without being bound to a particular theory, it is believed that more sensitive cognitive measures, particularly with challenging episodic memory measures, may detect very subtle cognitive impairment in amyloid-positive individuals. In certain embodiments criteria include, but are not limited to, self-complaint of memory decline or other subtle neurobehavioral changes.

As indicated above, subjects/patients amenable to prophylactic methods described herein include individuals at risk of disease (e.g., a pathology characterized by amyloid plaque formation such as MCI) but not showing symptoms, as well as subjects presently showing certain symptoms or markers. It is known that the risk of MCI and later Alzheimer's disease generally increases with age. Accordingly, in asymptomatic subjects with no other known risk factors, in certain embodiments, prophylactic application is contemplated for subjects over 50 years of age, or subjects over 55 years of age, or subjects over 60 years of age, or subjects over 65 years of age, or subjects over 70 years of age, or subjects over 75 years of age, or subjects over 80 years of age, in particular to prevent or slow the onset or ultimate severity of mild cognitive impairment (MCI), and/or to slow or prevent the progression from MCI to early stage Alzheimer's disease (AD).

In certain embodiments, the methods described herein present methods are especially useful for individuals who do have a known genetic risk of Alzheimer's disease (or other amyloidogenic pathologies), whether they are asymptomatic or showing symptoms of disease. Such individuals include those having relatives who have experienced MCI or AD (e.g., a parent, a grandparent, a sibling), and those whose risk is determined by analysis of genetic or biochemical markers. Genetic markers of risk toward Alzheimer's disease include, for example, mutations in the APP gene, particularly mutations at position 717 and positions 670 and 671 referred to as the Hardy and Swedish mutations respectively (see Hardy (1997) Trends. Neurosci., 20: 154-159). Other markers of risk include mutations in the presenilin genes (PS1 and PS2), family history of AD, having the familial Alzheimer's disease (FAD) mutation, the APOE ε4 allele, hypercholesterolemia or atherosclerosis. Further susceptibility genes for the development of Alzheimer's disease are reviewed, e.g., in Sleegers, et al. (2010) Trends Genet. 26(2): 84-93.

In some embodiments, the subject is asymptomatic but has familial and/or genetic risk factors for developing MCI or Alzheimer's disease. In asymptomatic patients, treatment can begin at any age (e.g., 20, 30, 40, 50 years of age). Usually, however, it is not necessary to begin treatment until a patient reaches at least about 40, 50, 60 or 70 years of age.

In some embodiments, the subject is exhibiting symptoms, for example, of mild cognitive impairment (MCI) or Alzheimer's disease (AD). Individuals presently suffering from Alzheimer's disease can be recognized from characteristic dementia, as well as the presence of risk factors described above. In addition, a number of diagnostic tests are available for identifying individuals who have AD. These include measurement of CSF Tau, phospho-tau (pTau), A1342 levels and C-terminally cleaved APP fragment (APPneo). Elevated total-Tau (tTau), phospho-Tau (pTau), APPneo, soluble Aβ40, pTau/Aβ42 ratio and tTau/Aβ42 ratio, and decreased A1342 levels, Aβ42/Aβ40 ratio, Aβ42/Aβ38 ratio, sAPPα levels, sAPPα/sAPPβ ratio, sAPPα/Aβ40 ratio, and sAPPα/Aβ42 ratio signify the presence of AD. In some embodiments, the subject or patient is diagnosed as having MCI. Increased levels of neural thread protein (NTP) in urine and/or increased levels of α2-macroglobulin (α2M) and/or complement factor H (CFH) in plasma are also biomarkers of MCI and/or AD (see, e.g., Anoop et al. (2010) Int. J. Alzheimer's Dis.2010:606802).

In certain embodiments, subjects amenable to treatment may have age-associated memory impairment (AAMI), or mild cognitive impairment (MCI). The methods described herein are particularly well-suited to the prophylaxis and/or treatment of MCI and/or other pre-Alzheimer's conditions. In such instances, the methods can delay or prevent the onset of MCI, and or reduce one or more symptoms characteristic of MCI and/or delay or prevent the progression from MCI to early-, mid- or late-stage Alzheimer's disease or reduce the ultimate severity of the disease.

There is emerging evidence that magnetic resonance imaging can observe deterioration, including progressive loss of gray matter in the brain, from mild cognitive impairment to full-blown Alzheimer disease (see, e.g., Whitwell et al. (2008) Neurology 70(7): 512-520). A technique known as PiB PET imaging is used to clearly show the sites and shapes of beta amyloid deposits in living subjects using a CI 1 tracer that binds selectively to such deposits (see, e.g., Jack et al. (2008) Brain 131(Pt 3): 665-680).

Mild Cognitive Impairment (MCI)

In various embodiments the tropinol esters and related esters described herein are contemplated in the treatment and/or prophylaxis of age-related cognitive decline and/or in the treatment and/or prophylaxis of mild cognitive impairment (MCI). Mile cognitive impairment, also known as incipient dementia, or isolated memory impairment) is a diagnosis given to individuals who have cognitive impairments beyond that expected for their age and education, but that typically do not interfere significantly with their daily activities (see, e.g., Petersen et al. (1999) Arch. Neurol. 56(3): 303-308). It is considered in many instances to be a boundary or transitional stage between normal aging and dementia. Although MCI can present with a variety of symptoms, when memory loss is the predominant symptom it is termed “amnestic MCI” and is frequently seen as a risk factor for Alzheimer's disease (see, e.g., Grundman et al. (2004) Arch. Neurol. 61(1): 59-66; and on the internet at en.wikipedia.org/wiki/Mild cognitive impairment—cite note-Grundman-1). When individuals have impairments in domains other than memory it is often classified as non-amnestic single- or multiple-domain MCI and these individuals are believed to be more likely to convert to other dementias (e.g. dementia with Lewy bodies). There is evidence suggesting that while amnestic MCI patients may not meet neuropathologic criteria for Alzheimer's disease, patients may be in a transitional stage of evolving Alzheimer's disease; patients in this hypothesized transitional stage demonstrated diffuse amyloid in the neocortex and frequent neurofibrillary tangles in the medial temporal lobe (see, e.g., Petersen et al. (2006) Arch. Neurol. 63(5): 665-72).

The diagnosis of MCI typically involves a comprehensive clinical assessment including clinical observation, neuroimaging, blood tests and neuropsychological testing. In certain embodiments diagnostic criteria for MIC include, but are not limited to those described by Albert et al. (2011) Alzheimer's & Dementia. 1-10. As described therein, diagnostic criteria include (1) core clinical criteria that could be used by healthcare providers without access to advanced imaging techniques or cerebrospinal fluid analysis, and (2) research criteria that could be used in clinical research settings, including clinical trials. The second set of criteria incorporate the use of biomarkers based on imaging and cerebrospinal fluid measures. The final set of criteria for mild cognitive impairment due to AD has four levels of certainty, depending on the presence and nature of the biomarker findings.

In certain embodiments clinical evaluation/diagnosis of MCI involves: (1) Concern reflecting a change in cognition reported by patient or informant or clinician (i.e., historical or observed evidence of decline over time); (2) Objective evidence of Impairment in one or more cognitive domains, typically including memory (i.e., formal or bedside testing to establish level of cognitive function in multiple domains); (3) Preservation of independence in functional abilities; (4) Not demented; and in certain embodiments, (5) An etiology of MCI consistent with AD pathophysiological processes. Typically vascular, traumatic, medical causes of cognitive decline, are ruled out where possible. In certain embodiments, evidence of longitudinal decline in cognition is identified, when feasible. Diagnosis is reinforced by a history consistent with AD genetic factors, where relevant.

With respect to impairment in cognitive domain(s), there should be evidence of concern about a change in cognition, in comparison with the person's previous level. There should be evidence of lower performance in one or more cognitive domains that is greater than would be expected for the patient's age and educational background. If repeated assessments are available, then a decline in performance should be evident over time. This change can occur in a variety of cognitive domains, including memory, executive function, attention, language, and visuospatial skills. An impairment in episodic memory (i.e., the ability to learn and retain new information) is seen most commonly in MCI patients who subsequently progress to a diagnosis of AD dementia.

With respect to preservation of independence in functional abilities, it is noted that persons with MCI commonly have mild problems performing complex functional tasks which they used to perform shopping. They may take more time, be less efficient, and make more errors at performing such activities than in the past. Nevertheless, they generally maintain their independence of function in daily life, with minimal aids or assistance.

With respect to dementia, the cognitive changes should be sufficiently mild that there is no evidence of a significant impairment in social or occupational functioning. If an individual has only been evaluated once, change will be inferred from the history and/or evidence that cognitive performance is impaired beyond what would have been expected for that individual.

Cognitive testing is optimal for objectively assessing the degree of cognitive impairment for an individual. Scores on cognitive tests for individuals with MCI are typically 1 to 1.5 standard deviations below the mean for their age and education matched peers on culturally appropriate normative data (i.e., for the impaired domain(s), when available).

Episodic memory (i.e., the ability to learn and retain new information) is most commonly seen in MCI patients who subsequently progress to a diagnosis of AD dementia. There are a variety of episodic memory tests that are useful for identifying those MCI patients who have a high likelihood of progressing to AD dementia within a few years. These tests typically assess both immediate and delayed recall, so that it is possible to determine retention over a delay. Many, although not all, of the tests that have proven useful in this regard are wordlist learning tests with multiple trials. Such tests reveal the rate of learning over time, as well as the maximum amount acquired over the course of the learning trials. They are also useful for demonstrating that the individual is, in fact, paying attention to the task on immediate recall, which then can be used as a baseline to assess the relative amount of material retained on delayed recall. Examples of such tests include (but are not limited to: the Free and Cued Selective Reminding Test, the Rey Auditory Verbal Learning Test, and the California Verbal Learning Test. Other episodic memory measures include, but are not limited to: immediate and delayed recall of a paragraph such as the Logical Memory I and II of the Wechsler Memory Scale Revised (or other versions) and immediate and delayed recall of nonverbal materials, such as the Visual Reproduction subtests of the Wechsler Memory Scale-Revised I and II.

Because other cognitive domains can be impaired among individuals with MCI, it is desirable to examine domains in addition to memory. These include, but are not limited to executive functions (e.g., set-shifting, reasoning, problem-solving, planning), language (e.g., naming, fluency, expressive speech, and comprehension), visuospatial skills, and attentional control (e.g., simple and divided attention). Many clinical neuropsychological measures are available to assess these cognitive domains, including (but not limited to the Trail Making Test (executive function), the Boston Naming Test, letter and category fluency (language), figure copying (spatial skills), and digit span forward (attention).

As indicated above, genetic factors can be incorporated into the diagnosis of MCI. If an autosomal dominant form of AD is known to be present (i.e., mutation in APP, PS1, PS2), then the development of MCI is most likely the precursor to AD dementia. The large majority of these cases develop early onset AD (i.e., onset below 65 years of age).

In addition, there are genetic influences on the development of late onset AD dementia. For example, the presence of one or two ε4 alleles in the apolipoprotein E (APOE) gene is a genetic variant broadly accepted as increasing risk for late-onset AD dementia. Evidence suggests that an individual who meets the clinical, cognitive, and etiologic criteria for MCI, and is also APOE ε4 positive, is more likely to progress to AD dementia within a few years than an individual without this genetic characteristic. It is believed that additional genes play an important, but smaller role than APOE and also confer changes in risk for progression to AD dementia (see, e.g., Bertram et al. (2010) Neuron, 21: 270-281).

In certain embodiments subjects suitable for the prophylactic methods described herein (e.g., administration of the tropinol esters and/or related esters described herein) include, but need not be limited to subjects identified having one or more of the core clinical criteria described above and/or subjects identified with one or more “research criteria” for MCI, e.g., as described below.

“Research criteria” for the identification/prognosis of MCI include, but are not limited to biomarkers that increase the likelihood that MCI syndrome is due to the pathophysiological processes of AD. Without being bound to a particular theory, it is believed that the conjoint application of clinical criteria and biomarkers can result in various levels of certainty that the MCI syndrome is due to AD pathophysiological processes. In certain embodiments, two categories of biomarkers have been the most studied and applied to clinical outcomes are contemplated. These include “Aβ” (which includes CSF Aβ₄₂ and/or PET amyloid imaging) and “biomarkers of neuronal injury” (which include, but are not limited to CSF tau/p-tau, hippocampal, or medial temporal lobe atrophy on MRI, and temporoparietal/precuneus hypometabolism or hypoperfusion on PET or SPECT).

Without being bound to a particular theory, it is believed that evidence of both Aβ, and neuronal injury (either an increase in tau/p-tau or imaging biomarkers in a topographical pattern characteristic of AD), together confers the highest probability that the AD pathophysiological process is present. Conversely, if these biomarkers are negative, this may provide information concerning the likelihood of an alternate diagnosis. It is recognized that biomarker findings may be contradictory and accordingly any biomarker combination is indicative (an indicator) used on the context of a differential diagnosis and not itself dispositive. It is recognized that varying severities of an abnormality may confer different likelihoods or prognoses, that are difficult to quantify accurately for broad application.

For those potential MCI subjects whose clinical and cognitive MCI syndrome is consistent with AD as the etiology, the addition of biomarker analysis effects levels of certainty in the diagnosis. In the most typical example in which the clinical and cognitive syndrome of MCI has been established, including evidence of an episodic memory disorder and a presumed degenerative etiology, the most likely cause is the neurodegenerative process of AD. However, the eventual outcome still has variable degrees of certainty. The likelihood of progression to AD dementia will vary with the severity of the cognitive decline and the nature of the evidence suggesting that AD pathophysiology is the underlying cause. Without being bound to a particular theory it is believed that positive biomarkers reflecting neuronal injury increase the likelihood that progression to dementia will occur within a few years and that positive findings reflecting both Ab accumulation and neuronal injury together confer the highest likelihood that the diagnosis is MCI due to AD.

A positive Aβ biomarker and a positive biomarker of neuronal injury provide an indication that the MCI syndrome is due to AD processes and the subject is well suited for the methods described herein.

A positive Aβ biomarker in a situation in which neuronal injury biomarkers have not been or cannot be tested or a positive biomarker of neuronal injury in a situation in which Aβ biomarkers have not been or cannot be tested indicate an intermediate likelihood that the MCI syndrome is due to AD. Such subjects are believed to be is well suited for the methods described herein

Negative biomarkers for both Aβ and neuronal injury suggest that the MCI syndrome is not due to AD. In such instances the subjects may not be well suited for the methods described herein.

There is evidence that magnetic resonance imaging can observe deterioration, including progressive loss of gray matter in the brain, from mild cognitive impairment to full-blown Alzheimer disease (see, e.g., Whitwell et al. (2008) Neurology 70(7): 512-520). A technique known as PiB PET imaging is used to clearly show the sites and shapes of beta amyloid deposits in living subjects using a C11 tracer that binds selectively to such deposits (see, e.g., Jack et al. (2008) Brain 131(Pt 3): 665-680).

In certain embodiments, MCI is typically diagnosed when there is 1) Evidence of memory impairment; 2) Preservation of general cognitive and functional abilities; and 3) Absence of diagnosed dementia.

In certain embodiments MCI and stages of Alzheimer's disease can be identified/categorized, in part by Clinical Dementia Rating (CDR) scores. The CDR is a five point scale used to characterize six domains of cognitive and functional performance applicable to Alzheimer disease and related dementias: Memory, Orientation, Judgment & Problem Solving, Community Affairs, Home & Hobbies, and Personal Care. The necessary information to make each rating is obtained through a semi-structured interview of the patient and a reliable informant or collateral source (e.g., family member).

The CDR table provides descriptive anchors that guide the clinician in making appropriate ratings based on interview data and clinical judgment. In addition to ratings for each domain, an overall CDR score may be calculated through the use of an algorithm. This score is useful for characterizing and tracking a patient's level of impairment/dementia: 0=Normal; 0.5=Very Mild Dementia; 1=Mild Dementia; 2=Moderate Dementia; and 3=Severe Dementia. An illustrative CDR table is shown in Table 3.

TABLE 3
Illustrative clinical dementia rating (CDR) table.

| Impairment: | None | Questionable | Mild | Moderate | Severe |
|---|---|---|---|---|---|
| CDR: | 0 | 0.5 | 1 | 2 | 3 |
| Memory | No memory | Consistent | Moderate | Severe | Severe |
| | loss or slight | slight | memory loss; | memory | memory |
| | inconsistent | forgetfulness; | more marked | loss; only | loss; only |
| | forgetfulness | partial | for recent | highly | fragments |
| | | recollection | events; defect | learned | remain |
| | | of events' | interferes | material | |
| | | “benign” | with | retained; | |
| | | forgetfulness | everyday | new material | |
| | | | activities | rapidly lost | |
| Orientation | Fully | Fully | Moderate | Severe | Oriented to |
| | oriented | oriented | difficulty | difficulty | person only |
| | | except for | with time | with time | |
| | | slight | relationships; | relationships; | |
| | | difficulty | oriented for | usually | |
| | | with time | place at | disoriented | |
| | | relationships | examination; | to time, often | |
| | | | may have | to place. | |
| | | | geographic | | |
| | | | disorientation | | |
| | | | elsewhere | | |
| Judgment & | Solves | Slight | Moderate | Severely | Unable to |
| Problem | everyday | impairment | difficulty in | impaired in | make |
| Solving | problems & | in solving | handling | handling | judgments |
| | handles | problems, | problems, | problems, | or solve |
| | business & | similarities, | similarities | similarities | problems |
| | financial | and | and | and | |
| | affairs well; | differences | differences; | differences; | |
| | judgment | | social | social | |
| | good in | | judgment | judgment | |
| | relation to | | usually | usually | |
| | past | | maintained | impaired | |
| | performance | | | | |
| Community | Independent | Slight | Unable to | No pretense of independent | |
| Affairs | function at | impairment | function | function outside of home | |
| | usual level | in these | independently | Appears well | Appears too |
| | in job, | activities | at these | enough to be | ill to be |
| | shopping, | | activities | taken to | taken to |
| | volunteer, | | although may | functions | functions |
| | and social | | still be | outsde a | outside a |
| | groups | | engaged in | family home | family |
| | | | some; | | home. |
| | | | appears | | |
| | | | normal to casual | | |
| | | | inspection | | |
| Home and | Life at | Life at home, | Mild bit | Only simple | No |
| Hobbies | home, | hobbies, and | definite | chores | significant |
| | hobbies, and | intellectual | impairment | preserved; | function in |
| | intellectual | interests | of function at | very | home |
| | interests | slightly | home; more | restricted | |
| | well | impaired | difficult | interests, | |
| | maintained | | chores | poorly | |
| | | | abandoned; | maintained | |
| | | | more | | |
| | | | complicated | | |
| | | | hobbies and | | |
| | | | interests | | |
| | | | abandoned | | |

| | | | | |
|---|---|---|---|---|
| Personal | Fully capable of self-care | Needs | Requires | Requires |
| Care | | prompting | assistance in | much help |
| | | | dressing, | with |
| | | | hygiene, | personal |
| | | | keeping of | care; |
| | | | personal | frequent |
| | | | effects | incontinence |

A CDR rating of ˜0.5 or ˜0.5 to 1.0 is often considered clinically relevant MCI. Higher CDR ratings can be indicative of progression into Alzheimer's disease.

In certain embodiments administration of a multi-component formulation described herein alone, or in combination with one or more active agents described herein (e.g., tropisetron and analogs thereof, tropinol esters and related esters, etc.) is deemed effective when there is a reduction in the CSF of levels of one or more components selected from the group consisting of Tau, phospho-Tau (pTau), APPneo, soluble Aβ40, soluble Aβ42, and/or Aβ42/Aβ40 ratio, and/or when there is a reduction of the plaque load in the brain of the subject, and/or when there is a reduction in the rate of plaque formation in the brain of the subject, and/or when there is an improvement in the cognitive abilities of the subject, and/or when there is a perceived improvement in quality of life by the subject, and/or when there is a significant reduction in clinical dementia rating (CDR), and/or when the rate of increase in clinical dementia rating is slowed or stopped and/or when the progression from MCI to early stage AD is slowed or stopped.

In some embodiments, a diagnosis of MCI can be determined by considering the results of several clinical tests. For example, Grundman, et al., Arch Neurol (2004) 61:59-66, report that a diagnosis of MCI can be established with clinical efficiency using a simple memory test (paragraph recall) to establish an objective memory deficit, a measure of general cognition (Mini-Mental State Exam (MMSE), discussed in greater detail below) to exclude a broader cognitive decline beyond memory, and a structured clinical interview (CDR) with patients and caregivers to verify the patient's memory complaint and memory loss and to ensure that the patient was not demented. Patients with MCI perform, on average, less than 1 standard deviation (SD) below normal on nonmemory cognitive measures included in the battery. Tests of learning, attention, perceptual speed, category fluency, and executive function may be impaired in patients with MCI, but these are far less prominent than the memory deficit.

Alzheimer's Disease (AD).

In certain embodiments the active agent(s) (e.g., tropinol esters and related esters described herein, analogues, derivatives, or prodrugs thereof) and/or formulations thereof are contemplated for the treatment of Alzheimer's disease. In such instances the methods described herein are useful in preventing or slowing the onset of Alzheimer's disease (AD), in reducing the severity of AD when the subject has transitioned to clinical AD diagnosis, and/or in mitigating one or more symptoms of Alzheimer's disease.

In particular, where the Alzheimer's disease is early stage, the methods can reduce or eliminate one or more symptoms characteristic of AD and/or delay or prevent the progression from MCI to early or later stage Alzheimer's disease.

Individuals presently suffering from Alzheimer's disease can be recognized from characteristic dementia, as well as the presence of risk factors described above. In addition, a number of diagnostic tests are available for identifying individuals who have AD. Individuals presently suffering from Alzheimer's disease can be recognized from characteristic dementia, as well as the presence of risk factors described above. In addition, a number of diagnostic tests are available for identifying individuals who have AD. These include measurement of CSF Tau, phospho-tau (pTau), sAPPα, sAPPβ, Aβ40, A1342 levels and/or C terminally cleaved APP fragment (APPneo). Elevated Tau, pTau, sAPPβ and/or APPneo, and/or decreased sAPPα, soluble Aβ40 and/or soluble A1342 levels, particularly in the context of a differential diagnosis, can signify the presence of AD.

In certain embodiments subjects amenable to treatment may have Alzheimer's disease. Individuals suffering from Alzheimer's disease can also be diagnosed by Alzheimer's disease and Related Disorders Association (ADRDA) criteria. The NINCDS-ADRDA Alzheimer's criteria were proposed in 1984 by the National Institute of Neurological and Communicative Disorders and Stroke and the Alzheimer's Disease and Related Disorders Association (now known as the Alzheimer's Association) and are among the most used in the diagnosis of Alzheimer's disease (AD). McKhann, et al. (1984) Neurology 34(7): 939-44. According to these criteria, the presence of cognitive impairment and a suspected dementia syndrome should be confirmed by neuropsychological testing for a clinical diagnosis of possible or probable AD. However, histopathologic confirmation (microscopic examination of brain tissue) is generally used for a dispositive diagnosis. The NINCDS-ADRDA Alzheimer's Criteria specify eight cognitive domains that may be impaired in AD: memory, language, perceptual skills, attention, constructive abilities, orientation, problem solving and functional abilities). These criteria have shown good reliability and validity.

Baseline evaluations of patient function can made using classic psychometric measures, such as the Mini-Mental State Exam (MMSE) (Folstein et al. (1975) J. Psychiatric Research 12 (3): 189-198), and the Alzheimer's Disease Assessment Scale (ADAS), which is a comprehensive scale for evaluating patients with Alzheimer's Disease status and function (see, e.g., Rosen, et al. (1984) Am. J. Psychiatr., 141: 1356-1364). These psychometric scales provide a measure of progression of the Alzheimer's condition. Suitable qualitative life scales can also be used to monitor treatment. The extent of disease progression can be determined using a Mini-Mental State Exam (MMSE) (see, e.g., Folstein, et al. supra). Any score greater than or equal to 25 points (out of 30) is effectively normal (intact). Below this, scores can indicate severe (≦9 points), moderate (10-20 points) or mild (21-24 points) Alzheimer's disease.

Alzheimer's disease can be broken down into various stages including: 1) Moderate cognitive decline (Mild or early-stage Alzheimer's disease), 2) Moderately severe cognitive decline (Moderate or mid-stage Alzheimer's disease), 3) Severe cognitive decline (Moderately severe or mid-stage Alzheimer's disease), and 4) Very severe cognitive decline (Severe or late-stage Alzheimer's disease) as shown in Table 4.

TABLE 4
Illustrative stages of Alzheimer's disease

Moderate Cognitive Decline (Mild or early stage AD)
At this stage, a careful medical interview detects clear-cut deficiencies
in the following areas:
Decreased knowledge of recent events.
Impaired ability to perform challenging mental arithmetic. For example,
to count backward from 100 by 7s.
Decreased capacity to perform complex tasks, such as marketing,
planning dinner for guests, or paying bills and managing finances.
Reduced memory of personal history.
The affected individual may seem subdued and withdrawn, especially in
socially or mentally challenging situations.
Moderately severe cognitive decline (Moderate or mid-stage
Alzheimer's disease)
Major gaps in memory and deficits in cognitive function emerge. Some
assistance with day-to-day activities becomes essential. At this stage,
individuals may:
Be unable during a medical interview to recall such important details as
their current address, their telephone number, or the name of the college
or high school from which they graduated.
Become confused about where they are or about the date, day of the
week or season.
Have trouble with less challenging mental arithmetic; for example,
counting backward from 40 by 4s or trom 20 by 2s.
Need help choosing proper clothing for the season or the occasion.
Usually retain substantial knowledge about themselves and know their
own name and the names of their spouse or children.
Usually require no assistance with eating or using the toilet.
Severe cognitive decline (Moderately severe or mid-stage
Alzheimer's disease)
Memory difficulties continue to worsen, significant personality changes
may emerge, and affected individuals need extensive help with daily
activities. At this stage, individuals may:
Lose most awareness of recent experiences and events as well as of their
surroundings.
Recollect their personal history imperfectly, although they generally
recall their own name.
Occasionally forget the name of their spouse or primary caregiver but
generally can distinguish familiar from unfamiliar faces.
Need help getting dressed properly; without supervision, may make
such errors as putting pajamas over daytime clothes or shoes on
wrong feet.
Experience disruption of their normal sleep/waking cycle.
Need help with handling details of toileting (flushing toilet, wiping and
disposing of tissue properly).
Have increasing episodes of urinary or fecal incontinence.
Experience significant personality changes and behavioral symptoms,
including suspiciousness and delusions (for example, believing that their
caregiver is an impostor); hallucinations (seeing or hearing things that
are not really there); or compulsive, repetitive behaviors such as
hand-wringing or tissue shredding.
Tend to wander and become lost.
Very severe cognitive decline (Severe or late-stage Alzheimer's disease)
This is the final stage of the disease when individuals lose the ability to
respond to their environment, the ability to speak, and, ultimately, the
ability to control movement.
Frequently individuals lose their capacity for recognizable speech,
although words or phrases may occasionally be uttered.
Individuals need help with eating and toileting and there is general
incontinence.
Individuals lose the ability to walk without assistance, then the ability to
sit without support, the ability to smile, and the ability to hold their
head up.
Reflexes become abnormal and muscles grow rigid. Swallowing is
impaired.

In various embodiments administration of one or more agents described herein to subjects diagnosed with Alzheimer's disease is deemed effective when the there is a reduction in the CSF of levels of one or more components selected from the group consisting of Tau, phospho-Tau (pTau), APPneo, soluble Aβ40, soluble Aβ42, and/or and Aβ42/Aβ40 ratio, and/or when there is a reduction of the plaque load in the brain of the subject, and/or when there is a reduction in the rate of plaque formation in the brain of the subject, and/or when there is an improvement in the cognitive abilities of the subject, and/or when there is a perceived improvement in quality of life by the subject, and/or when there is a significant reduction in clinical dementia rating (CDR) of the subject, and/or when the rate of increase in clinical dementia rating is slowed or stopped and/or when the progression of AD is slowed or stopped (e.g., when the transition from one stage to another as listed in Table 4 is slowed or stopped).

In certain embodiments subjects amenable to the present methods generally are free of a neurological disease or disorder other than Alzheimer's disease. For example, in certain embodiments, the subject does not have and is not at risk of developing a neurological disease or disorder such as Huntington's Disease, and/or Parkinson's disease, and/or schizophrenia, and/or psychosis.

In various embodiments, the effectiveness of treatment can be determined by comparing a baseline measure of a parameter of disease before administration of the multi-component formulation, alone or in conjunction with the other active agent(s) described herein (e.g., tropisetron and analogs thereof, tropinol esters and related esters, etc.) is commenced to the same parameter one or more time points after the multi-component formulation and/or additional active agent(s) have been administered. One illustrative parameter that can be measured is a biomarker (e.g., a peptide oligomer) of APP processing. Such biomarkers include, but are not limited to increased levels of sAPPα, p3 (Aβ 17-42 or Aβ 17-40), βAPPβ, soluble Aβ40, and/or soluble Aβ42 in the blood, plasma, serum, urine, mucous or cerebrospinal fluid (CSF). Detection of increased levels of sAPPα and/or p3, and decreased levels of βAPPβ and/or APPneo is an indicator that the treatment is effective. Conversely, detection of decreased levels of sAPPα and/or p3, and/or increased levels of βAPPβ, APPneo, Tau or phospho-Tau (pTau) is an indicator that the treatment is not effective.

Another parameter to determine effectiveness of treatment is the level of amyloid plaque deposits in the brain. Amyloid plaques can be determined using any method known in the art, e.g., as determined by CT, PET, PIB-PET and/or MRI.

In various embodiments administration of the multi-component formulation alone or in conjunction with one or more other active agent(s) described herein can result in a reduction in the rate of plaque formation, and even a retraction or reduction of plaque deposits in the brain. Effectiveness of treatment can also be determined by observing a stabilization and/or improvement of cognitive abilities of the subject. Cognitive abilities can be evaluated using any art-accepted method, including for example, Clinical Dementia Rating (CDR), the mini-mental state examination (MMSE) or Folstein test, evaluative criteria listed in the DSM-IV (Diagnostic and Statistical Manual of Mental Disorders, Fourth Edition) or DSM-V, and the like.

In certain embodiments, the monitoring methods can entail determining a baseline value of a measurable biomarker or parameter (e.g., amyloid plaque load or cognitive abilities) in a subject before administering a dosage of the multi-component formulation and optionally one or more pharmaceuticals, and comparing this biomarker or parameter with a value for the same measurable biomarker or parameter after treatment.

In other methods, a control value (e.g., a mean and standard deviation) of the measurable biomarker or parameter is determined for a control population. In certain embodiments, the individuals in the control population have not received prior treatment and do not have AD, MCI, nor are at risk of developing AD or MCI. In such cases, if the value of the measurable biomarker or clinical parameter approaches the control value, then treatment is considered efficacious. In other embodiments, the individuals in the control population have not received prior treatment and have been diagnosed with AD or MCI. In such cases, if the value of the measurable biomarker or clinical parameter approaches the control value, then treatment is considered inefficacious.

In other methods, a subject who is not presently receiving treatment but has undergone a previous course of treatment is monitored for one or more of the biomarkers or clinical parameters to determine whether a resumption of treatment is required. The measured value of one or more of the biomarkers or clinical parameters in the subject can be compared with a value previously achieved in the subject after a previous course of treatment. Alternatively, the value measured in the subject can be compared with a control value (mean plus standard deviation/ANOVA) determined in population of subjects after undergoing a course of treatment. Alternatively, the measured value in the subject can be compared with a control value in populations of prophylactically treated subjects who remain free of symptoms of disease, or populations of therapeutically treated subjects who show amelioration of disease characteristics. In such cases, if the value of the measurable biomarker or clinical parameter approaches the control value, then treatment is considered efficacious and need not be resumed. In all of these cases, a significant difference relative to the control level (e.g., more than a standard deviation) is an indicator that treatment should be resumed in the subject.

In various embodiments the tissue sample for analysis is typically blood, plasma, serum, urine, mucous or cerebrospinal fluid from the subject.

### Compounding, Kits/Packaging Systems, and Administration.

In various embodiments, the multi-component formulations may be provided alone or in combination with one or more additional pharmaceuticals (e.g., tropiestron or analogs thereof, tropinol esters and other related esters, e.g. as described above). In certain embodiments, a combination formulation is contemplated wherein the pharmaceutical (e.g., tropiestron, a tropinol ester, and the like) is formulated with one or more components comprising the multi-component formulations described herein. In certain embodiments, one or more additional pharmaceuticals (e.g., tropiestron or the other pharmaceuticals described above) is provided along with the multi-component formulations described herein in a packing system or kit.

Compounding Multi-Component Formulations.

In certain embodiments, the components of the multi-component formulations may each be formulated individually, for example, in unit dosage forms such that a subject is able to select the particular individual components and the quantities thereof to suit its particular needs.

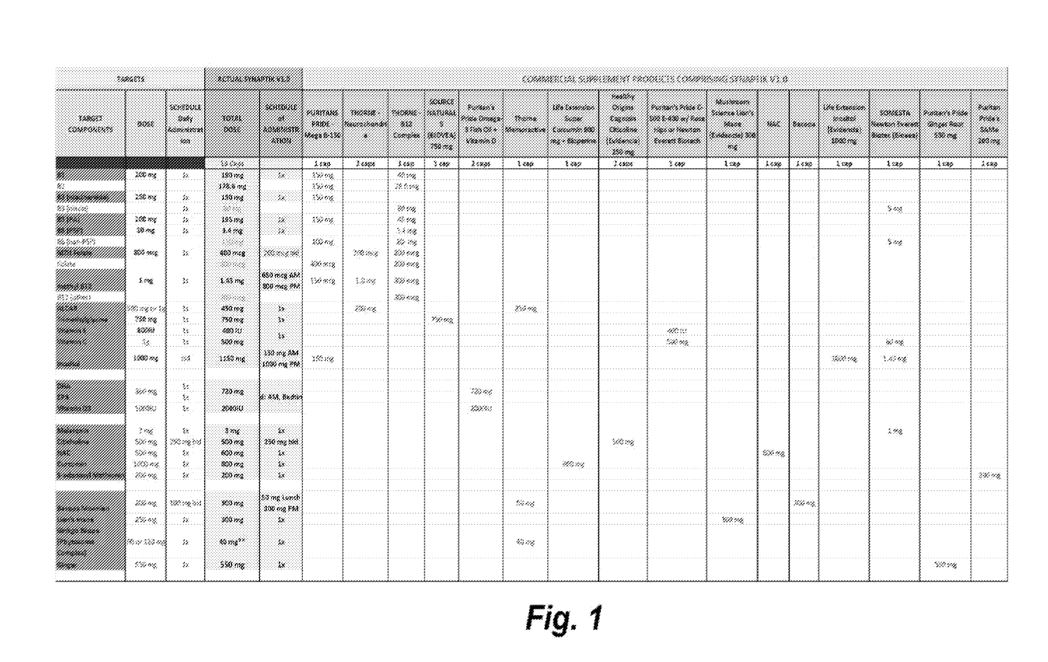

Alternatively, some of the components of the multi-component formulation may be formulated as one composition, so as to facilitate and encourage patient compliance. For example, in certain embodiments, a B complex formulation can be provided that includes as one component vitamin B1, B3, B5, B6, methyl folate, B12 and acetyl L-carnitine (see, e.g., FIG. 1), while omega-3 fatty acids are provided in a second component, and combinations of various herbs (e.g., Bacopa monnieri, lion's mane, Gingko biloba, and ginger) are provided as a third component.

It will be recognized that in this manner, delivery of a complete multi-component formulation can be accomplished by the use of combinations of commercially available dietary supplements. For example, FIG. 1 illustrates one formulation of a Synaptik™ multi-component formulation that incorporates vitamins (vitamin B1, vitamin B3 (niacinamide), vitamin B5 (PA), vitamin B6 (P5P), methyl (MTH) folate, methyl B12, ALCAR (acetyl caranitine), vitamin E, vitamin C, vitamin D3), carbohydrates (inositol), amino acids (trimethylglycine, N-acetyl cysteine (NAC), and S-adenosyl methionine), omega-3 fatty acids (DHA and EPA), lipid/phospholipid (citicholine), melatonin, a phenol (curcumin), and various herbs (herbs (e.g., Bacopa monnieri, lion's mane, Gingko biloba (phytosome complex), and ginger). As shown in FIG. 1, this multi-component formulation can be achieved with a combination of commercially available supplements, e.g., PURITANS PRIDE® Mega B-150, THORNE® Neurochondria, THORNE® B12 Complex, SOURCE NATURALS® (BIOVEA), PURITAN'S PRIDE® Omega-3 Fish Oil plus Vitamin D, THORNE® MEMORACTIVE®, LIFE EXTENSION® Super Curcumin plus Bioperine, HEALTHY ORIGINS COGNIZIN® CITICOLINE (Evidencia), PURITAN'S PRIDE® C-500 E-400 with Rose Hips, MUSHROOM SCIENCE® Lion's Mane (Evidencia), NAC, Bacopa, LIFE EXTENSION® Inositol (Evidencia), SOMESTA® NEWTON EVERETT BIOTEC® (BIOVEA), PURITAN'S PRIDE® Ginger Root, PURITAN'S PRIDE® SAMe).

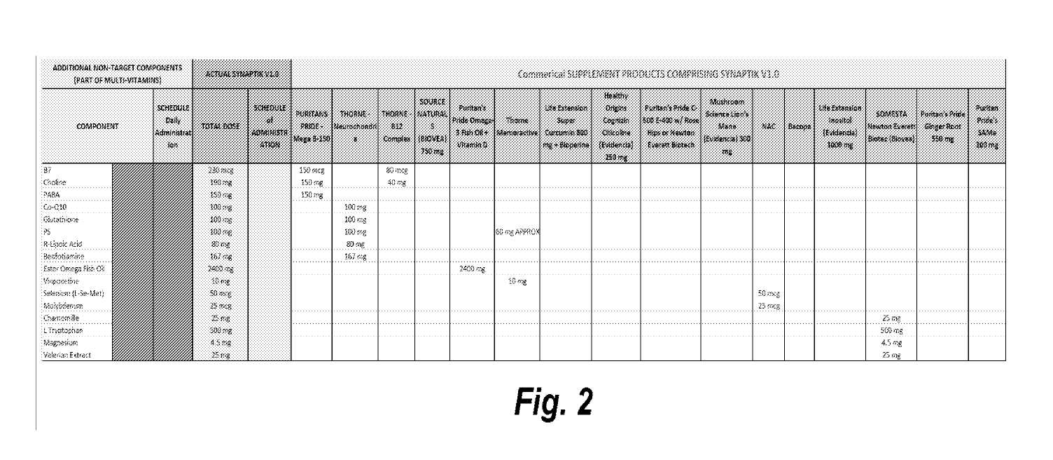

In particular embodiments, using combinations of commercial products to achieve the multi-component formulations contemplated herein typically introduces additional components. Thus, for example, FIG. 2 illustrates nutritional supplements that would be added (over and above) the desired multi-component formulation using the combinations of products shown in FIG. 1. In certain embodiments, the introduction of such additional components may not be desired, e.g., where the combination pushes particular components above the recommended maximum daily dosage.

Accordingly, in certain embodiments, the agents comprising the multi-component formulation may be compounded into one or more “unit dosage” forms. Techniques for formulation and administration of drugs may be found in “Remington: The Science and Practice of Pharmacy.” 21st Edition. Philadelphia, Pa. Lippincott Williams & Wilkins. 2005’, which is incorporated herein by reference in its entirety. The nature of the formulation will depend on the intended route(s) of administration. Suitable routes of administration may, for example, include oral, rectal, transmucosal (e.g., transnasal), intestinal, parenteral delivery, including intramuscular, subcutaneous and intramedullary injections as well as intrathecal, intravenous, intranasal, or intraocular injections. Preferably, the multi-component formulations described herein are administered orally.

The multi-component formulations described herein or subsets of components comprising the multi-component formulations may be manufactured by processes well known in the art, e.g., by means of conventional mixing, dissolving, granulating, dragee-making, levigating, emulsifying, encapsulating, entrapping or lyophilizing processes.

Thus, for example, in certain embodiments, multi-component formulations described herein or subsets of components comprising the multi-component formulations are formulated for oral administration. For oral administration, suitable formulations can be readily formulated by combining the active agent(s) with pharmaceutically acceptable carriers suitable for oral delivery well known in the art. Such carriers enable the active agent(s) described herein to be formulated as tablets, pills, dragees, caplets, lozenges, gelcaps, capsules, liquids, gels, syrups, slurries, suspensions and the like, for oral ingestion by a patient/subject to be treated. For oral solid formulations such as, for example, powders, capsules and tablets, suitable excipients can include fillers such as sugars (e.g., lactose, sucrose, mannitol and sorbitol), cellulose preparations (e.g., maize starch, wheat starch, rice starch, potato starch, gelatin, gum tragacanth, methyl cellulose, hydroxypropylmethyl-cellulose, sodium carboxymethylcellulose), synthetic polymers (e.g., polyvinylpyrrolidone (PVP)), granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques. The preparation of enteric-coated particles is disclosed for example in U.S. Pat. Nos. 4,786,505 and 4,853,230.

In certain embodiments, the multi-component formulations described herein or subsets of components comprising the multi-component formulations prepared for oral use can be made using a solid excipient, optionally grinding the resulting mixture, and processing the mixture of granules, after adding suitable auxiliaries if desired, to obtain tablets or dragee cores. Suitable excipients include, but are not limited to, particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). As indicated above, if desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Formulations that can also be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. Formulations for oral administration should typically be in dosages suitable for the chosen route of administration

Multi-component formulations described herein or subsets of components comprising the multi-component formulations for administration by inhalation, the active agent(s) are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator may be formulated containing a powder mix of the compound and a suitable powder base such as lactose or starch.

In various embodiments, the active agent(s) can be formulated in rectal compositions such as suppositories or retention enemas, e.g., containing conventional suppository bases such as cocoa butter or other glycerides. Methods of formulating active agents for rectal delivery are well known to those of skill in the art (see, e.g., Allen (2007) Suppositories, Pharmaceutical Press) and typically involve combining the active agents with a suitable base (e.g., hydrophilic (PEG), lipophilic materials such as cocoa butter or Witepsol W45), amphiphilic materials such as Suppocire AP and polyglycolized glyceride, and the like). The base is selected and compounded for a desired melting/delivery profile.

In certain embodiments, the multi-component formulations described herein or subsets of components comprising the multi-component formulations are formulated for systemic administration (e.g., as an injectable) in accordance with standard methods well known to those of skill in the art. Systemic formulations include, but are not limited to, those designed for administration by injection, e.g. subcutaneous, intravenous, intramuscular, intrathecal or intraperitoneal injection, as well as those designed for transdermal, transmucosal oral or pulmonary administration. For injection, the active agents described herein can be formulated in aqueous solutions, preferably in physiologically compatible buffers such as Hanks solution, Ringer's solution, or physiological saline buffer and/or in certain emulsion formulations. The solution(s) can contain formulatory agents such as suspending, stabilizing and/or dispersing agents. In certain embodiments, the active agent(s) can be provided in powder form for constitution with a suitable vehicle, e.g., sterile pyrogen-free water, before use. For transmucosal administration, and/or for blood/brain barrier passage, penetrants appropriate to the barrier to be permeated can be used in the formulation. Such penetrants are generally known in the art. Injectable formulations and inhalable formulations are generally provided as a sterile or substantially sterile formulation.

In addition to the formulations described previously, the multi-component formulations described herein or subsets of components comprising the multi-component formulations) may also be formulated as a depot preparations. Such long acting formulations can be administered by implantation (for example subcutaneously or intramuscularly) or by intramuscular injection. Thus, for example, the active agent(s) may be formulated with suitable polymeric or hydrophobic materials (for example as an emulsion in an acceptable oil) or ion exchange resins, or as sparingly soluble derivatives, for example, as a sparingly soluble salt.

In certain embodiments, multi-component formulations described herein or subsets of components comprising the multi-component formulations described herein can be provided as a “concentrate”, e.g., in a storage container (e.g., in a premeasured volume) ready for dilution, or in a soluble capsule ready for addition to a volume of water, alcohol, hydrogen peroxide, or other diluent.

In certain embodiments, the multi-component formulations described herein or subsets of components comprising the multi-component formulations may also be provided as food additives. Food additives include, for example, any liquid or solid material that is intended to be added to a food product. This material can, for example, include an agent having a distinct taste and/or flavor or a physiological effect (e.g., the multicomponent formulations described herein or subsets of the components comprising such formulations). In various embodiments, the multi-component formulations described herein or subsets of components comprising the multi-component formulations described herein can be added to a variety of food products.

As used herein, the phrase “food product” describes a material consisting essentially of protein, carbohydrate and/or fat, that is used in the body of an organism to sustain growth, repair and vital processes and to furnish energy. Food products may also contain supplementary substances such as minerals, vitamins and condiments. The phrase “food product” as used herein further includes a beverage adapted for human or animal consumption.

A food product containing the multi-component formulations described herein or subsets of components comprising the multi-component formulations described herein can also include additional additives such as, for example, certain antioxidants, sweeteners, flavorings, colors, preservatives, nutritive additives such as vitamins and minerals, amino acids (i.e. essential amino acids), emulsifiers, pH control agents such as acidulants, hydrocolloids, antifoams and release agents, flour improving or strengthening agents, raising or leavening agents, gases and chelating agents, the utility and effects of which are well-known in the art.

The foregoing methods and forms of compounding and/or providing the multi-component formulations described herein or subsets of components comprising the multi-component formulations described herein are intended to be illustrative and not limiting. Using the teachings provided herein, other methods of formulating and/or delivering the multi-component formulations described herein or subsets of components comprising the multi-component formulations described herein will be available to one of skill in the art.

Administration/Treatment Schedules.

The multi-component formulations can be administered on treatment schedules determined by the treatment modality of the pharmaceutical(s) (e.g., tropisetron, tropisetron analogs, tropinol esters and related esters, galangin, galangin prodrugs, and the like) if administered, and/or by the number and nature of the components comprising the multi-component formulation, and/or by the nature and severity of the pathology (e.g., pre-Alzheimer's disease, mild cognitive impairment, early stage Alzheimer's disease, late stage Alzheimer's disease, age-related dementia, Parkinson's disease, Huntington's disease, multiple sclerosis, amyotrophic lateral sclerosis (ALS or Lou Gehrig's Disease), prion diseases, Creutzfeldt-Jakob disease, Lewy body disease, Friedreich's ataxia, stroke, genetic brain disorders, etc.). The specific amount/dosage regimen will vary depending on the weight, gender, age and health of the individual; the formulation, the biochemical nature, bioactivity, bioavailability and the side effects of the pharmaceuticals (e.g., tropiestron, galangin, etc. if administered), and the number and/or components of the multi-component formulation.

One illustrative, but non limiting treatment schedule using commercially available supplements to provide the multi-component formulation, optionally in conjunction with a pharmaceutical (e.g., tropisetron designated F03) is shown in Table 5.

TABLE 5
Illustrative treatment schedule for administration of a multi-
component formulation (formulated as shown in FIG. 1) in combination
with an additional pharmaceutical (e.g., tropisetron (F03)).

| | DAILY ADMINISTRATION SCHEDULE | | | |
|---|---|---|---|---|
| | (Number of Tablets/Capsules) | | | |
| | AM | LUNCH | DINNER | BEDTIME |
| Pharma- | F03 | | F03 | |
| ceutical | | | | |
| Supple- | PURITAN'S | THORNE | THORNE B-12 | SUNESTA |
| ment | PRIDE | Memoractive | Complex (1) | Melatonin |
| | Mega B-150 | (1) | | (1) |
| | (1) | | | |
| | THORNE | LE | THORNE | PP |
| | Neurochondria | Curcumin- | Neurochondria | Omega3 - |
| | (1) | Bioperine | (1) | Vit D (1) |
| | | (1) | | |
| | BIOVEA | PP C-500/ | Bacopa | |
| | ALCAR | E-400 (1) | monnieri | |
| | (1) | | (1) | |
| | PP Omega 3 - | MS Lion's | LE Inositol | |
| | Vit D | Mane (1) | (1) | |
| | (1) | | | |
| | HO Cognizin | Ginger | HO Cognizin | |
| | (1) | (1) | (1) | |
| | | SAMe | | |
| | | (1) | | |
| Total | 6 | 6 | 6 | 2 |
| Capsule | | | | |
| Count | | | | |

This treatment schedule is intended to be illustrative and non-limiting. Using the teaching provided herein, other treatment schedules will be available to one of skill in the art.

Kits and Packaging Systems.

In certain embodiments, the components of the multi-component formulations may each be formulated individually, for example, in unit dosage forms such that a subject is able to select the particular individual components and the quantities thereof to suit its particular needs. Even, when formulated individually, patient/subject compliance can be improved and convenience afforded by providing the components in an integrated kit or packaging system. For example, where the components are individually formulated a kit can comprise one or more packages containing some or all of the components.

Alternatively, some of the components of the multi-component formulation may be formulated as one composition and/or bundled together in various packaging systems e.g., a pack or dispenser device, such as an FDA approved kit, that can contain one or more unit dosage forms comprising the multi-component formulation and when present, one or more additional pharmaceuticals (e.g., tropisetron).

The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the multi-component formulations described and/or claimed herein and/or additional pharmaceuticals, formulated in a compatible pharmaceutical carrier may also be prepared, placed in an appropriate container, and labeled for treatment of an indicated condition, as further detailed above.

The packaging system or kit can be constructed to facilitate administration on a particular treatment schedule. In one, non-limiting and illustrative embodiment, FIG. 3 shows a blister pack packaging system structured to provide a pharmaceutical (e.g., tropiestron) with the multi-component formulation according to the treatment schedule shown below in Table 5. As illustrated therein the multi-component formulation is delivered by administering 5 formulation (supplement) tablets in the morning (e.g., at breakfast), 6 formulation tablets at noon (e.g., at lunch), 5 formulation tablets in the evening (e.g., at dinner) and two formulation tablets at bedtime. The pharmaceutical (e.g., tropisetron) is administered twice daily as shown. These combinations of tablets can be provided in blisterpack rows labeled with the time of administration as shown in FIG. 3.

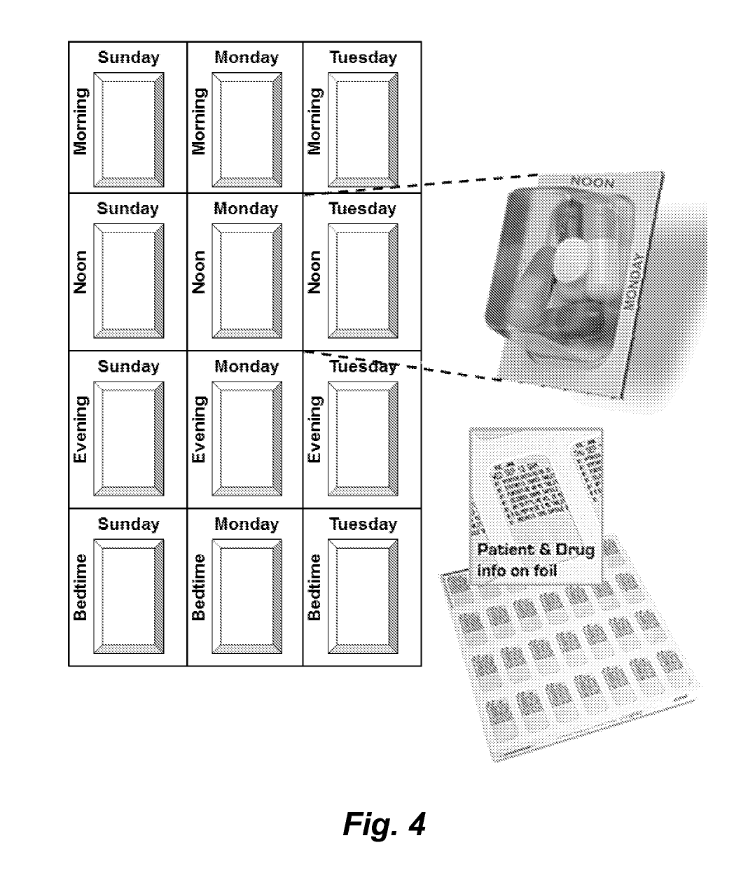

In various embodiments, the packaging system need not contain each unit dosage formulation within a single package. As illustrated in FIG. 4, the multi-component formulation and one or more additional pharmaceuticals can be provided in multi-component packages using perforated heat seal punch card packaging (see, e.g., MTS Medication Technologies). As illustrated, the packaging provides a perforatable system comprising a plurality of labeled (e.g., date/time labeled) containers that the components that are to be consumed at the indicated time. The card's inside cover provides the space to clearly label each prescription and associated instructions. The perforated card allows the patient to take their medications with them in a smaller container.

It will be appreciated that these kits/packaging systems are intended to be illustrative and not limiting. Using the teachings provided herein, numerous alternative packaging/dispensing systems will be available to provide the multi-component formulations as described herein.

In addition, the packaging systems/kits optionally include labeling and/or instructional materials providing directions (i.e., protocols) for the practice of the methods or use of the “therapeutics” or “prophylactics” described and/or claimed herein. Illustrative instructional materials describe the use of the multi-component formulations described and/or claimed herein alone, or in combination with one or more pharmaceuticals in the treatment or prophylaxis of a neurodegenerative pathology. In certain embodiments, the instructional materials may also, optionally, teach preferred dosages/therapeutic regiment, counter indications and the like. In this regard, it is noted that certain herbal supplements are counter indicated when the subject is administered certain neuroactive pharmaceuticals (e.g., MAOI inhibitors, and the like).

While the instructional materials typically comprise written or printed materials they are not limited to such. Any medium capable of storing such instructions and communicating them to an end user is contemplated. Such media include, but are not limited to electronic storage media (e.g., magnetic discs, tapes, cartridges, chips), optical media (e.g., CD ROM), and the like. Such media may include addresses to internet sites that provide such instructional materials.

It is understood that the examples and embodiments, described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A multi-component formulation comprising:
a first component comprising one or more vitamins selected from the group consisting of one or more B vitamins, vitamin C, vitamin D, vitamin E, co-enzyme Q10, vitamin K, and folate;
a second component comprising one or more elements selected from the group consisting of selenium, lithium, magnesium, and molybdenum;
a third component comprising one or more omega-3 fatty acids; and
a fourth component comprising one or more amino acids selected from the group consisting of trimethylglycine, N-acetyl cysteine, S-adenosyl methionine, L-tryptophan, and glutathione.

2. The formulation of claim 1, wherein said formulation further comprises a fifth component comprising one or more herbs selected from the group consisting of lion's main (Hericium), Bacopa monnieri, Ginkgo biloba, honokiol, magnolia extract, rosemary extract, ashwagandha, blueberry extract, bilberry extract, ginger, he shou wu, rhodiola, reishi, saffron, and daffodil.

3. The formulation according to any one of claims 1-2, wherein said formulation further comprises a sixth component comprising one or more active agents selected from the group consisting of pregnenolone, galangin, vinpocetine, astaxanthin, and huperzine A.

4. The formulation according to any one of claims 1-3, wherein said formulation further comprises a seventh component comprising a natural phenol.

5. The formulation according to any one of claims 1-4, wherein said formulation further comprises an eighth component comprising a lipid or phospholipid.

6. The formulation according to any one of claims 1-5, wherein said formulation further comprises a ninth component comprising a carbohydrate.

7. The formulation according to any one of claims 1-6, wherein said B vitamins comprise one or more vitamins selected from the group consisting of vitamin B1, vitamin B2, vitamin B3, vitamin B5, vitamin B6, vitamin B7, vitamin B12, vitamin Bt (Carnitine), vitamin Benfotiamine, and vitamin Bx (PABA).

8. The formulation according to any one of claims 1-7, wherein said vitamins comprise one or more vitamins selected from the group consisting of thiamine, nicotinamide, pantothenic acid, pyridoxal 5-phosphate, B12, vitamin C, vitamin E, vitamin K, and folate.

9. The formulation according to any one of claims 1-7, wherein said vitamins comprise thiamine, nicotinamide, pantothenic acid, pyridoxal 5-phosphate, B12, vitamin C, vitamin E, vitamin K, and folate.

10. The formulation according to any one of claims 1-7, wherein said vitamins comprise thiamine, nicotinamide, pantothenic acid, pyridoxine or pyridoxal 5-phosphate, B12 (preferably hydroxocobalamin or methylcobalamin), vitamin C, vitamin E, vitamin K, and folate.

11. The formulation according to any one of claims 1-10, wherein one or more elements comprises lithium.

12. The formulation according to any one of claims 1-11, wherein said omega-3 fatty acid comprises one or more a fatty acids selected from the group consisting of docosahexaenoic acid, and eicosapentaenoic acid.

13. The formulation according to any one of claims 1-11, wherein said omega-3 fatty acid comprises docosahexaenoic acid.

14. The formulation according to any one of claims 1-13, wherein said one or more amino acids comprise one or more amino acids selected from the group consisting of trimethyl glycine, N-acetyl cysteine, and S-adenosyl methionine.

15. The formulation according to any one of claims 1-13, wherein said one or more amino acids comprise trimethyl glycine, N-acetyl cysteine, and S-adenosyl methionine.

16. The formulation according to any one of claims 2-13, wherein said one or more herbs comprise one or more herbs selected from the group consisting of Withania somnifera (ashwagandha), Reishi, Rhodiola, Lion's Mane (Hericium Erinaceous), Bacopa monnieri, Ginkgo biloba, Honokiol, and ginger.

17. The formulation according to any one of claims 2-13, wherein said one or more herbs comprise Lion's Mane (Hericium erinaceus), Bacopa monnieri, Ginkgo biloba, Withania somnifera (ashwagandha), Reishi, Rhodiola, Honokiol, and Ginger.

18. The formulation according to any one of claims 3-17, wherein said one or more active agents comprise one or more active agents selected from the group consisting of pregnenolone, and galangin.

19. The formulation according to any one of claims 3-17, wherein said active agents comprise pregnenolone, and galangin.

20. The formulation according to any one of claims 4-19, wherein said natural phenols comprise a cucuminoid.

21. The formulation according to any one of claims 4-19, wherein said natural phenols comprise cucumin and/or turmeric.

22. The formulation according to any one of claims 5-21, wherein said lipid or phospholipid comprise one or more lipids or phospholipids selected from the group consisting of CDP-choline, Phosphatidyl choline, Choline, Phosphatidyl Serine, and Lipoic Acid.

23. The formulation according to any one of claims 5-21, wherein said lipid or phospholipid comprises choline.

24. The formulation according to any one of claims 6-23, wherein said carbohydrate comprises inositol.

25. The formulation according to any one of claims 1-5, wherein said formulation comprises at least four agents selected from the group consisting of vitamin B1, vitamin B5, nicotinamide, vitamin B6, vitamin B12, carnitine, vitamin C, vitamin D, vitamin E, vitamin K, folate, selenium, lithium, Docosahexaenoic Acid, eisopantanoic acid, choline, Trimethylglycine, L-Tryptophan, N-Acetyl-Cysteine, S-Adenosyl Methionine (SAMe), Melatonin, Pregnenolone, Galangin, Lion's Mane (Hericium Erinaceous), Bacopa monnieri, Ginkgo biloba, Withania somnifera (ashwagandha), Reishi, Rhodiola, Honokiol, and ginger, wherein said at least four different agents comprise at least four different components.

26. The formulation of claim 25, wherein said formulation comprises at least five different agents selected from said group and said at least five different agents comprise at least five different components.

27. The formulation of claim 25, wherein said formulation comprises at least six different agents selected from said group and said at least six different agents comprise at least six different components.

28. The formulation of claim 25, wherein said formulation comprises at least seven different agents selected from said group and said at least seven different agents comprise at least seven different components.

29. The formulation of claim 25, wherein said formulation comprises at least eight different agents selected from said group and said at least eight different agents comprise at least eight different components.

30. The formulation of claim 3, wherein said formulation comprises:
said first component wherein said first component comprises vitamin B1, and/or vitamin B5, and/or nicotinamide and/or vitamin B6, and/or vitamin B12, and/or carnitine, and/or vitamin C, and/or vitamin E, and/or vitamin K, and/or folate;
said second component wherein said second component comprises selenium and/or lithium;
said third component wherein said third component comprises an omega-3 fatty acid;
said fourth component wherein said fourth component comprises trimethylglycine, and/or N-acetyl cysteine, and/or S-adenosyl methionine;
said fifth component wherein said fifth component comprises Lion's Mane, and/or Bacopa monnieri, and/or Ginkgo biloba, and/or Withania somnifera (ashwagandha), and/or Reishi, and/or Rhodiola, and/or Honokiol; and
said sixth component wherein said sixth component comprises pregnenolone, and/or galangin.

31. The formulation of claim 30, wherein:
said first component comprises vitamin B1, vitamin B5, nicotinamide, vitamin B6, vitamin B12, carnitine, vitamin C, vitamin E, vitamin K, and folate;
said second component comprises selenium and/or lithium;
said third component comprises docosahexaenoic acid, and/or eisopentanoic Acid;
said fourth component comprises trimethylglycine, N-acetyl cysteine, and S-adenosyl methionine;
said fifth component comprises Lion's Mane, Bacopa monnieri, Ginkgo biloba, Withania somnifera (ashwagandha), Reishi, Rhodiola, and Honokiol; and
said sixth component comprises melatonin, pregnenolone, and galangin.

32. The formulation according to any one of claims 30-31, wherein said formulation further comprises said seventh component, wherein said seventh component comprises a cucuminoid.

33. The formulation according to any one of claims 30-32, wherein said formulation further comprises said eighth component, wherein said eighth component comprises a lipid or phospholipid.

34. The formulation of claim 33, wherein said lipid or phospholipid comprises choline.

35. The formulation according to any one of claims 30-34, wherein said formulation further comprises said ninth component, wherein said ninth component comprises inosotol.

36. The formulation according to any one of claims 1-35 wherein:
vitamin B1, when present, comprises at least about 2.5 mg;
nicotinamide, when present, comprises at least 50 mg;
vitamin B5, when present, comprises at least 50 mg;
vitamin B6, when present, comprises at least 5 mg;
vitamin B12, when present, comprises at least about 0.1 mg;
carnitine, when present, comprises at least about 100 mg;
vitamin C, when present, comprises at least about 100 mg;
vitamin D, when present, comprises at least about 1000 IU;
vitamin E, when present, comprises at least about 50 mg;
vitamin K, when present, comprises at least about 10 mg;
folate, when present, comprises at least about 0.2 mg;
selenium, when present, comprises at least about 25 μg;
lithium, when present, comprises at least about 1 mg;
inosotol, when present, comprises at least about 500 mg;
docosahexaenoic acid, when present, comprises at least about 0.25 g;
eicosapentanoic acid, when present, comprises at least about 0.25 g;
choline, when present, comprises at least about 0.5 g;
trimethylglycine, when present, comprises at least about 120 mg;
N-acetyl-cysteine, when present, comprises at least about 200 mg;
S-adenosyl methionine, when present, comprises at least about 100 mg;
a curcuminoid, when present, comprises at least about 250 mg;
pregnenolone, when present, comprises at least about 2 mg;
galangin, when present, comprises at least about 200 mg;
Lion's Mane, when present, comprises at least about 250 mg;
Bacopa monnieri, when present, comprises at least about 50 mg;
Ginkgo biloba, when present, comprises at least about 20 mg;
Honokiol, when present, comprises at least about 200 mg; and
Ginger, when present, comprises at least about 100 mg.

37. The formulation of claim 1-36, wherein:
vitamin B1, when present, ranges from about 100 to about 750 mg;
vitamin B5, when present, ranges from about 25 to about 150 mg;
vitamin B6, when present, ranges from about 5 to about 50 mg;
vitamin B12, when present, ranges from about 0.1 mg to about 3 mg;
acetyl-L-carnitine (ALCAR), when present, ranges from about 250 mg to about 2000 mg;
vitamin C, when present, ranges from about 100 mg to about 1000 mg
vitamin D, when present, ranges from about 1000 IU to about 4000 IU;
vitamin E, when present, ranges from about 50 mg to about 1500 mg;
vitamin K, when present, ranges from about 10 mg to about 200 mg;
folate, when present, ranges from about 0.2 mg to about 1.5 mg;
selenium, when present, ranges from about 25 μg to about 500 μg;
lithium, when present, ranges from about 1 mg to about 20 mg;
inosotol, when present, ranges from about 0.25 mg to about 1.5 mg;
docosahexaenoic acid, when present, ranges from about 0.25 g to about 1.5 g;
eicosapentaenoic, when present, ranges from about 0.25 g to about 1.5 g;
choline, when present, ranges from about 0.5 g to about 3 g;
trimethylglycine, when present, ranges from about 120 mg to about 1000 mg;
N-acetyl-cysteine, when present, ranges from about 200 mg to about 1000 mg;
S-adenosyl methionine, when present, ranges from about 100 mg to about 600 mg;
a curcuminoid, when present, ranges from about 500 mg to about 4000 mg;
pregnenolone, when present, ranges from about 2 mg to about 5 mg;
galangin, when present, ranges from about 200 mg to about 8000 mg;
Lion's Mane, when present, ranges from about 250 mg to about 2000 mg;
Bacopa monnieri, when present, ranges from about 50 mg to about 600 mg;
Ginkgo biloba, when present, ranges from about 20 mg to about 200 mg;
Honokiol, when present, ranges from about 1 mg to about 1000 mg active ingredient; and
Ginger, when present, ranges from about 100 mg to about 1000 mg.

38. The formulation according to any one of claims 1-35, wherein:
vitamin B1 is present and ranges from about 100 to about 750 mg;
vitamin B5 is present and ranges from about 25 to about 150 mg;
vitamin B6 is present and ranges from about 5 to about 50 mg;
vitamin B12 is present and ranges from about 0.1 mg to about 3 mg;
acetyl-L-carnitine (ALCAR) is present and ranges from about 250 mg to about 2000 mg;
vitamin C is present and ranges from about 100 mg to about 1000 mg;
vitamin D is present and ranges from about 1000 IU to about 4000 IU;
vitamin E is present and ranges from about 50 mg to about 1500 mg;
vitamin K is present and ranges from about 10 mg to about 200 mg;
folate is present and ranges from about 0.2 mg to about 1.5 mg;
selenium is present and ranges from about 25 μg to about 500 μg;
lithium is present and ranges from about 1 mg to about 20 mg;
inosotol is present and ranges from about 0.25 mg to about 1.5 mg;
docosahexaenoic acid is present and ranges from about 0.25 g to about 1.5 g;
eicosapentaenoic acid is present and ranges from about 0.25 g to about 1.5 g;
choline is present and ranges from about 0.5 g to about 3 g;
trimethylglycine is present and ranges from about 120 mg to about 1000 mg;
N-acetyl-cysteine is present and ranges from about 200 mg to about 1000 mg;
S-adenosyl methionine is present and ranges from about 100 mg to about 600 mg;
a curcuminoid is present and ranges from about 500 mg to about 4000 mg;
pregnenolone is present and ranges from about 2 mg to about 5 mg;
galangin is present and ranges from about 200 mg to about 1000 mg;
Lion's Mane is present and ranges from about 250 mg to about 2000 mg;
Bacopa monnieri is present and ranges from about 50 mg to about 600 mg;
Ginkgo biloba is present and ranges from about 20 mg to about 200 mg;
Honokiol is present and ranges from about 1 mg to about 1000 mg; and
Ginger is present and ranges from about 100 mg to about 1000 mg.

39. The formulation according to any one of claims 1-38, wherein said components are contained in single packaging system.

40. The formulation according to any one of claims 1-39, wherein two or more of said components are encapsulated in separate capsules, vials, or tablets.

41. The formulation according to any one of claims 1-40, wherein fluid components are encapsulated separately from solid components.

42. The formulation according to any one of claims 1-39, wherein all of said components are provided in a single combined formulation.

43. A method of slowing the rate of decrease in neurological function, or delaying the onset of a decrease in neurological function in a mammal, said method comprising administering, or causing to be administered, to said mammal a multi-component formulation according to any one of claims 1-38 in an amount sufficient to slow the rate of decrease in neurological function or to delay the onset of a decrease in neurological function in said mammal.

44. The method of claim 43, wherein said mammal is a mammal that has a neurological disorder.

45. The method of claim 43, wherein said mammal is a mammal that has been identified as at risk for a neurological disorder.

46. The method of claim 43, wherein said mammal is a normal healthy mammal and said decrease in neurological function is an age related decrease in neurological function.

47. The method of claim 43, wherein said mammal is a normal healthy mammal and said decrease in neurological function is a stress-induced decrease in neurological function.

48. A method of improving neurological function or in a mammal, said method comprising administering, or causing to be administered, to said mammal a multi-component formulation according to any one of claims 1-38 in an amount sufficient to improve neurological function.

49. The method of claim 48, wherein said mammal is a mammal that has a neurological disorder.

50. The method of claim 48, wherein said mammal is a mammal that has been identified as at risk for a neurological disorder.

51. The method of claim 48, wherein said mammal with no neurological disorder.

52. A method of normalizing neurological function to optimize treatment for a neurological disorder in a mammal, said method comprising administering, or causing to be administered, to said mammal a multi-component formulation according to any one of claims 1-38 in an amount sufficient to:
improve cognitive function as measured by a standard neuropsychological cognitive test in a subject with abnormal cognition or in a subject with normal cognition; and/or
to prevent or delay progression of symptoms of neurodegeneration.

53. A method of preventing or delaying the onset of a pre-Alzheimer's condition and/or cognitive dysfunction, and/or ameliorating one or more symptoms of a pre-Alzheimer's condition and/or cognitive dysfunction, or preventing or delaying the progression of a pre-Alzheimer's condition or cognitive dysfunction to Alzheimer's disease in a mammal, said method comprising:
Administering, or causing to be administered, to said mammal a multi-component formulation according to any one of claims 1-38 in an amount sufficient to slow the rate of decrease in neurological function or to prevent or delay the onset of a pre-Alzheimer's condition and/or cognitive dysfunction, and/or to ameliorate one or more symptoms of a pre-Alzheimer's condition and/or cognitive dysfunction, and/or to prevent or delay the progression of a pre-Alzheimer's condition or cognitive dysfunction to Alzheimer's disease in said mammal.

54. The method according to any one of claims 43-53, wherein the neurological function comprises one or more functions selected from the group consisting of memory, cognition, concentration, gross motor control, and fine motor control.

55. The method according to any one of claims 52-54, wherein an improvement in neurological function is characterized by, or associated with, a reduction in the mammal's CSF of levels of one or more components selected from the group consisting of total-Tau (tTau), phospho-Tau (pTau), APPneo, soluble Aβ40, pTau/Aβ42 ratio and tTau/Aβ42 ratio, and/or an increase in the mammal's CSF of levels of one or more components selected from the group consisting of Aβ40/Ab42 ratio, Aβ38/Ab42 ratio, sAPPα, sAPPα/sAPPβ ratio, sAPPα/Aβ40 ratio, or sAPPα/Aβ42 ratio.

56. The method according to any one of claims 52-54, wherein slowing the rate of a decrease in neurological function is characterized by, or associated with, a stabilization or a reduction in the mammal's CSF of levels of one or more components selected from the group consisting of total-Tau (tTau), phospho-Tau (pTau), APPneo, soluble Aβ40, pTau/Aβ42 ratio and tTau/Aβ42 ratio, and/or a stabilization or an increase in the mammal's CSF of levels of one or more components selected from the group consisting of Aβ40/Ab42 ratio, Aβ38/Ab42 ratio, sAPPα, sAPPα/sAPPβ ratio, sAPPα/Aβ40 ratio, or sAPPα/Aβ42 ratio.

57. The method according to any one of claims 52-56, wherein all components of said formulation are administered to said mammal at least once a day.

58. The method according to any one of claims 52-57, wherein said mammal is diagnosed with a neurological disorder selected from the group consisting of pre-Alzheimer's disease, mild cognitive impairment, early stage Alzheimer's disease, late stage Alzheimer's disease, age-related dementia, Parkinson's disease, Huntington's disease, Multiple Sclerosis, Amyotrophic Lateral Sclerosis (ALS or Lou Gehrig's Disease), Prion Diseases, Creutzfeldt-Jakob disease, Lewy Body disease, Friedreich's Ataxia, Stroke, Genetic Brain Disorders, Schizophrenia, ADHD, Autism, Aspergers syndrome, and Downs syndrome.

59. The method according to any one of claims 52-57, wherein said mammal is determined to be at risk for a neurological disorder selected from the group consisting of pre-Alzheimer's disease, mild cognitive impairment, early stage Alzheimer's disease, late stage Alzheimer's disease, age-related dementia, Parkinson's disease, Huntington's disease, Multiple Sclerosis, Amyotrophic Lateral Sclerosis (ALS or Lou Gehrig's Disease), Prion Diseases, Creutzfeldt-Jakob disease, Lewy Body disease, Friedreich's Ataxia, Stroke, Genetic Brain Disorders, Schizophrenia, ADHD, Autism, Aspergers syndrome, and Downs syndrome.

60. The method according to any one of claims 52-57, wherein said neurological disorder comprises MCI.

61. The method according to any one of claims 52-57, wherein said neurological disorder comprises Alzheimer's disease.

62. The method according to any one of claims 52-61, wherein said mammal is a human.

63. The method of claim 62, wherein said mammal is a human diagnosed as having or as at risk for said neurological disorder.

64. The method of claim 62, wherein said mammal is a human diagnosed as having or as at risk for MCI.

65. The method of claim 62, wherein said mammal is a human diagnosed as having or as at risk for said Alzheimer's disease.

66. A method of enhancing the efficacy of an agent for the treatment and/or prophylaxis of a neurological disorder in a mammal, said method comprising administering in conjunction with said agent a multi-component formulation according to any one of claims 1-39.

67. The method of claim 66, wherein all components of said formulation are administered to said mammal at least once a day.

68. The method according to any one of claims 66-67, wherein said neurological disorder comprises a disorder selected from the group consisting pre-Alzheimer's disease, mild cognitive impairment, early stage Alzheimer's disease, late stage Alzheimer's disease, age-related dementia, Parkinson's disease, Huntington's disease, Sclerosis, Amyotrophic Lateral Sclerosis (ALS or Lou Gehrig's Disease), Prion Diseases, Creutzfeldt-Jakob disease, Lewy Body disease, Friedreich's Ataxia, Stroke, Genetic Brain Disorders, Schizophrenia, ADHD, Autism, Aspergers syndrome, and Downs syndrome.

69. The method of claim 68, wherein said neurological disorder comprises MCI or another pre-Alzheimer's condition.

70. The method of claim 68, wherein said neurological disorder comprises Alzheimer's disease.

71. The method according to any one of claims 66-70, wherein said mammal is a human.

72. The method of claim 71, wherein said mammal is a human diagnosed as having or as at risk for said neurological disorder.

73. The method of claim 71, wherein said mammal is a human diagnosed as having or as at risk for MCI.

74. The method of claim 71, wherein said mammal is a human diagnosed as having or as at risk for said Alzheimer's disease.

75. The method according to any one of claims 66-74, wherein said agent comprises a therapeutic or prophylactic agent selected from the group consisting of a tropisetron analog, disulfiram, a disulfiram analog, honokiol, a honokiol analog, nimetazepam, a nimetazepam analog, tropinol-esters, ADDN-1351, TrkA kinase inhibitors, donepezil, rivastigmine, galantamine, tacrine, memantine, solanezumab, bapineuzmab, alzemed, flurizan, ELND005, valproate, semagacestat, rosiglitazone, phenserine, cernezumab, dimebon, egcg, gammagard, PBT2, PF04360365, NIC5-15, bryostatin-1, AL-108, nicotinamide, EHT-0202, BMS708163, NP12, lithium, ACC001, AN1792, ABT089, NGF, CAD106, AZD3480, SB742457, AD02, huperzine-A, EVP6124, PRX03140, PUFA, HF02, MEM3454, TTP448, PF-04447943, GSK933776, MABT5102A, talsaclidine, UB311, begacestat, R1450, PF3084014, V950, E2609, MK0752, CTS21166, AZD-3839, LY2886721, CHF5074, an anti-inflammatory, dapsone, an anti-TNF antibody, and a statin.

76. The method of claim 75, wherein said agent is tropisetron.

77. A method for the treatment or prophylaxis of a neurological/neurodegenerative disorder in a mammal, said method comprising administering, or causing to be administered, to said mammal:
one or more agents for the treatment or prophylaxis of a neurological disorder; and
a formulation according to any one of claims 1-39.

78. The method of claim 77, wherein all components of said formulation are administered to said mammal at least once a day.

79. The method according to any one of claims 77-78, wherein said neurological and neurodegenerative disorder comprises a disorder selected from the group consisting of pre-Alzheimer's disease, mild cognitive impairment, early stage Alzheimer's disease, late stage Alzheimer's disease, age-related dementia, Parkinson's disease, Huntington's disease, Multiple Sclerosis, Amyotrophic Lateral Sclerosis (ALS or Lou Gehrig's Disease), Prion Diseases, Creutzfeldt-Jakob disease, Lewy Body disease, Friedreich's Ataxia, Stroke, Genetic Brain Disorders, Schizophrenia, ADHD, Autism, Aspergers syndrome, and Downs syndrome.

80. The method of claim 79, wherein said neurological disorder comprises pre-Alzheimer's disease.

81. The method of claim 79, wherein said neurological disorder comprises MCI.

82. The method of claim 79, wherein said neurological disorder comprises Alzheimer's disease.

83. The method according to any one of claims 77-82, wherein said mammal is a human.

84. The method according to any one of claims 77-83, wherein said mammal is a human having or determined to be at risk for MCI.

85. The method of any one of claims 77-84, wherein said administration delays or prevents the progression of MCI to Alzheimer's disease.

86. The method of any one of claims 77-85, wherein the mammal is at risk of developing Alzheimer's disease.

87. The method of claim 86, wherein the mammal has a familial risk for having Alzheimer's disease.

88. The method of claim 86, wherein the mammal has a familial Alzheimer's disease (FAD) mutation.

89. The method of claim 86, wherein the mammal has the APOE 84 allele.

90. The method of any one of claims 77-89, wherein the mammal is free of and does not have genetic risk factors of Parkinson's disease or schizophrenia.

91. The method of any one of claims 77-89, wherein the mammal is not diagnosed as having or at risk for Parkinson's disease or schizophrenia.

92. The method of any one of claims 77-89, wherein the mammal does not have a neurological disease or disorder other than Alzheimer's disease.

93. The method of any one of claims 77-89, wherein the mammal is not diagnosed as having or at risk for a neurological disease or disorder other than Alzheimer's disease.

94. The method of any one of claims 77-89, wherein the mammal does not have a neurological disease or disorder other than MCI.

95. The method of any one of claims 77-89, wherein the mammal is not diagnosed as having or at risk for a neurological disease or disorder other than MCI.

96. The method of any one of claims 77-95, wherein the method results in a reduction in the CSF of levels of one or more components selected from the group consisting of total-Tau (tTau), phospho-Tau (pTau), APPneo, soluble Aβ40, pTau/Aβ42 ratio and tTau/Aβ42 ratio, and/or an increase in the CSF of levels of one or more components selected from the group consisting of Aβ40/Aβ42 ratio, Aβ38/Aβ42 ratio, sAPPα, sAPPα/sAPPβ ratio, sAPPα/Aβ40 ratio, and sAPPα/Aβ42 ratio.

97. The method of any one of claims 77-95, wherein the method produces a reduction of the plaque load in the brain of the mammal.

98. The method of any one of claims 77-95, wherein the method produces a reduction in the rate of plaque formation in the brain of the mammal.

99. The method of any one of claims 77-95 wherein the mitigation comprises an improvement in the cognitive abilities of the mammal.

100. The method of any one of claims 77-95, wherein the method produces an improvement in, a stabilization of, or a reduction in the rate of decline of the clinical dementia rating (CDR) of the mammal.

101. The method of any one of claims 77-95, wherein the mammal is a human and the method produces a perceived improvement in quality of life by the human.

102. The method of any one of claims 77-101, wherein the administering is over a period of at least three weeks.

103. The method of any one of claims 77-101, wherein the administering is over a period of at least 6 months.

104. The method according to any one of claims 77-103, wherein said agent comprises a therapeutic or prophylactic agent selected from the group consisting of a tropisetron analog, disulfiram, a disulfiram analog, honokiol, a honokiol analog, nimetazepam, a nimetazepam analog, tropinol-esters, ADDN-1351, TrkA kinase inhibitors, donepezil, rivastigmine, galantamine, tacrine, memantine, solanezumab, bapineuzmab, alzemed, flurizan, ELND005, valproate, semagacestat, rosiglitazone, phenserine, cernezumab, dimebon, egcg, gammagard, PBT2, PF04360365, NIC5-15, bryostatin-1, AL-108, nicotinamide, EHT-0202, BMS708163, NP12, lithium, ACC001, AN1792, ABT089, NGF, CAD106, AZD3480, SB742457, AD02, huperzine-A, EVP6124, PRX03140, PUFA, HF02, MEM3454, TTP448, PF-04447943, GSK933776, MABT5102A, talsaclidine, UB311, begacestat, R1450, PF3084014, V950, E2609, MK0752, CTS21166, AZD-3839, LY2886721, CHF5074, an anti-inflammatory, dapsone, an anti-TNF antibody, and a statin.

105. The method of claim 104, wherein said agent is tropisetron.

106. The method of any one of claims any one of claims 77-105, wherein an acetylcholinesterase inhibitor is not administered in conjunction with said compound.

107. The method of claim 106, wherein the acetylcholinesterase inhibitor is selected from the group consisting of tacrine-ipidacrine, galantamine, donepezil, icopezil, zanapezil, rivastigmine, Namenda, huperzine A, phenserine, physostigmine, neostigmine, pyridostigmine, ambenonium, demarcarium, edrophonium, ladostigil and ungeremine and metrifonate.

108. A kit for the treatment or prophylaxis of a neurological disorder, said kit comprising a packaging system containing one or more agents for the treatment or prophylaxis of said neurological disorder; and a formulation according to any one of claims 1-39.

109. The kit of claim 108, wherein the components of said formulation are contained in a first packaging system and said one or more agents are contained in a second packaging system.

110. The kit according to any one of claims 108-109, wherein two or more of the components of said formulation components are encapsulated in separate capsules, vials, or tablets.

111. The kit according to any one of claims 108-110, wherein fluid components of said formulation are encapsulated separately from solid components.

112. The kit of claim 108, wherein all of said components of said formulation are provided in a single combined formulation.

113. The kit according to any one of claims 108-112, wherein said agent comprises a therapeutic or prophylactic agent selected from the group consisting of a tropisetron analog, disulfiram, a disulfiram analog, honokiol, a honokiol analog, nimetazepam, a nimetazepam analog, tropinol-esters, ADDN-1351, TrkA kinase inhibitors, donepezil, rivastigmine, galantamine, tacrine, memantine, solanezumab, bapineuzmab, alzemed, flurizan, ELND005, valproate, semagacestat, rosiglitazone, phenserine, cernezumab, dimebon, egcg, gammagard, PBT2, PF04360365, NIC5-15, bryostatin-1, AL-108, nicotinamide, EHT-0202, BMS708163, NP12, lithium, ACC001, AN1792, ABT089, NGF, CAD106, AZD3480, SB742457, AD02, huperzine-A, EVP6124, PRX03140, PUFA, HF02, MEM3454, TTP448, PF-04447943, GSK933776, MABT5102A, talsaclidine, UB311, begacestat, R1450, PF3084014, V950, E2609, MK0752, CTS21166, AZD-3839, LY2886721, CHF5074, an anti-inflammatory, dapsone, an anti-TNF antibody, and a statin.

114. The kit of claim 113, wherein said agent is tropisetron.
